(19) United States
(12) Patent Application Publication
(10) Pub. No.: US 2024/0271081 A1
(43) Pub. Date: Aug. 15, 2024

(54) OLEAGINOUS YEAST YARROWIA LIPOLYTICA SUR2 MUTANT STRAIN AND METHOD FOR PRODUCING AND SECRETING, ONTO CELL SURFACES, SPHINGOID-BASED PRECURSORS, SPHINGOSINE AND SPHINGOLIPIDS BY USING SAME

(71) Applicant: CHUNG ANG UNIVERSITY INDUSTRY ACADEMIC COOPERATION FOUNDATION, Seoul (KR)

(72) Inventors: Hyun Ah KANG, Seoul (KR); Hye Yun MOON, Seoul (KR); Haeeun PARK, Seoul (KR); Seo Hyeon SHIN, Goyang-si, Gyeonggi-do (KR)

(73) Assignee: CHUNG ANG UNIVERSITY INDUSTRY ACADEMIC COOPERATION FOUNDATION, Seoul (KR)

(21) Appl. No.: 18/416,978

(22) Filed: Jan. 19, 2024

Related U.S. Application Data
(63) Continuation of application No. PCT/KR2022/010607, filed on Jul. 20, 2022.

(30) Foreign Application Priority Data
Jul. 22, 2021 (KR) 10-2021-0096309
Jul. 19, 2022 (KR) 10-2022-0088719

(51) Int. Cl. C12N 1/18; C12P 13/00; C12P 17/06; C12R 1/85

(52) U.S. Cl. CPC C12N 1/185; C12P 13/001; C12P 17/06; C12N 2500/35; C12R 2001/85

(57) ABSTRACT

The present invention relates to an oleaginous yeast Yarrowia lipolytica sur2 mutant strain and a method for producing and secreting, onto cell surfaces, sphingoid-based precursors, sphingosine and sphingolipids by using same. It has been identified that when a ceramidase gene is additionally introduced into a Ylsur2 deficient mutant strain, the secretion and production of dihydrosphingosine and sphingosine are increased and, further, it is has been identified that when a Ylsld1sur2 double mutant strain in which a SLD1 gene is additionally deficient in a Ylsur2 deficient mutant strain is prepared, growth recovery and sphingosine and human-type glucosylceramide secretion and production are increased.

### CROSS REFERENCE TO RELATED APPLICATIONS

This application is a Continuation of PCT International Patent Application No. PCT/KR2022/010607 filed on Jul. 20, 2022, which claims priority to Korean Patent Application Nos. 10-2021-0096309 filed on Jul. 22, 2021 and 10-2022-0088719 filed on Jul. 19, 2022 which are all hereby incorporated by reference in their entirety.

### SEQUENCE LISTING

This application contains a Sequence Listing submitted via USPTO Patent Center and hereby incorporated by reference in its entirety. The Sequence Listing is named SEQCRF_2280-503.xml, created on Jan. 18, 2024, and 60,259 bytes in size.

### BACKGROUND OF THE INVENTION

The present disclosure relates to an oleaginous yeast Yarrowia lipolytica sur2 mutant strain and a cell surface secretion and production method of a sphingoid-based precursor, sphingosine, and sphingolipid using the same, and more specifically, to a method of preparing a mutant strain to have an inactivated SUR2 gene encoding dihydrosphingosine C4-hydroxylase that is involved in phytosphingosine production in Y. lipolytica, and secreting and producing dihydrosphingosines, various sphingosine precursors, and glucosylceramides, sphingosine-based lipids, onto a cell surface with high efficiency even without acetylation, by using the mutant strain. Furthermore, the present disclosure relates to a method of preparing a strain that secretes and produces sphingosine by amplifying expression of a ceramidase gene or additionally inactivating an SLD1 gene using a Y. lipolytica sur2 mutant strain as a parent strain.

Sphingolipids refer to a group of lipids derived from sphingoid bases, such as sphingosine, are mainly present in cell membranes of animals, plants, and microorganisms. Ceramide, a type of sphingolipid, is a molecule made up of a sphingoid base and a fatty acid. Ceramide is a main lipid component of the stratum corneum, which is an upper layer of the skin, and topical application of a composition including ceramide may improve a barrier function and a moisture-retention property of the skin. In addition, sphingoid bases, including sphingosine and dihydrosphingosine, are known to mediate a number of physiological effects, such as suppression of protein kinase C (PKC) and are included in compositions for anti-inflammatory and antimicrobial activity or in skin- and hair-strengthening compositions.

Synthesis of sphingolipids begins when the amino acid serine and palmitate, one of the lipids, are combined, and a palmitoyl group binds to the serine by a serine palmitoyltransferase (SPT). Enzymatic activity of SPT mainly depends on a concentration of saturated fatty acids, which are the substrate, to regulate a degree of biosynthesis of sphingolipids. Afterwards, dihydrosphingosine is produced by 3-keto-dihydrosphingosine reductase. This process is common in microorganisms, plants, and animals. Afterwards, plants synthesize phytosphingosine and phytoceramide to finally produce glycosyl inositol phospho ceramide (GIPC), along with the conversion of dihydrosphingosine to ceramide and glucosylceramide (GlcCer). In contrast, animals synthesize mostly ceramide starting with dihydrosphingosine to produce sphingomyelin, galactosylceramide, gangliosides, and glucosylceramides. In the case of yeast, there is a very large difference in the sphingolipid biosynthesis between Saccharomyces cerevisiae and many other unconventional yeasts. Starting with dihydrosphingosine, S. cerevisiae strains do not have a DESI gene that encodes sphingolipid desaturase and produce only phytosphingosine and phytoceramide to synthesize mannosyl inositol phospho ceramide (MIPC) only, while many other yeasts have the DESI gene and produce not only phytoceramide but also ceramide, starting with dihydrosphingosine to synthesize MIPC, GIPC, and GlcCer (FIG. 1).

As such, dihydrosphingosine is a major substance that is common in the production process of sphingolipid of all living things, thereby synthesizing various types of sphingolipids using the same. Therefore, while sphingolipid and dihydrosphingosine are attracting attention as important ingredients in many cosmetic industries, raw materials of these sphingolipids have been derived from animals such as cattle, but due to the problem concerning infection, plant-based sphingolipids such as those from rice, wheat, soybeans, and potatoes are now mainly used. However, since a trace amount of sphingolipids exists in animals and plants, extraction and purification are difficult and expensive, such that the development of new production technologies that may overcome the issue is required, and thus a technology for mass production of sphingolipid using microorganisms is attracting attention as a new strategy.

In the case of traditional yeast S. cerevisiae, it was revealed that dihydrosphingosine increases in the cell of mutant strains that are deficient of SUR2, a gene encoding dihydrosphingosine C4-hydroxylase that introduces the —OH group to the fourth carbon. However, a production level was shown to be only 346 pmol per 1 mg of intracellular protein, corresponding to approximately 10 ug per gram of biomass. This is a very low level that is insufficient for efficient production in commercial processes. Through amplification of TSC10, a gene encoding 3-keto dihydrosphingosine reductase involved in dihydrosphingosine synthesis by targeting wild-type S. cerevisiae, a recombinant yeast whose synthesis level for dihydrosphingosine and ceramide is raised to 9.8 mg per 1 g was described. In addition, a recombinant yeast strain has been prepared, which produces human ceramide (ceramide-NS) by introducing a human-derived DESI gene into the SUR2 deficient strain. Unlike ceramide, sphingoid bases are known to cause apoptosis, which may cause problems in the case of recombinant yeasts that overproduce multiple sphingoid bases, including sphingosine. Pichia ciferrii exclusively produces phytosphingosine and its acetylated derivatives, the production levels of which have been found to be much higher than those required for de novo biosynthesis of sphingolipid. In other words, using superior acetylase, sphingoid bases such as phytosphingosine may be secreted to bypass the problem. In fact, P. ciferrii SUR2 mutant strains obtained using syringomycin E have been identified to overproduce its acetylated derivatives with accumulation of dihydrosphingosine. In addition, P. ciferrii, unlike S. cerevisiae, has DESI to convert dihydrosphingosine into ceramide. As overexpression of the sphingolipid desaturase gene DESI and introduction of the additional mouse-derived alkaline ceramidase gene have made sphingosine detachable from ceramide, it was reported that a yield of sphingosine and its acetylated derivatives became at a level of 240 mg/L.

Since Yarrowia lipolytica yeast, an oleaginous yeast, is capable of well utilizing hydrophobic substrates, it may grow well with cheap substrates such as alkanes and also retain lipid droplets, which are composed of more than 20% fat content in the cell. These lipid droplets store mostly triacylglycerides (TAGs) and also include components such as ergosterol, which is known to be absorbed or released in and out of the cell to regulate the amount. Y. lipolytica may follow the same process as microorganisms, plants, and animals up to the synthesis of dihydrosphingosine, and synthesize a number of complex sphingolipids, including inositol, similar to characteristics of other yeasts. Owing to the DESI gene present in Y. lipolytica, it is possible to produce several ceramides and glucosylceramides that have sphingosine as the basic backbone. In the case of the Y. lipolytica strain, acetylation of sphingoid bases such as phytosphingosine was observed by introducing P. ciferrii acetylase genes ATF2 and SLI1 into wild-type strains, and additional deletion of the LCB4 gene encoding a sphingoid long-chain base kinase that adds a phosphate group to the sphingoid base has shown that productivity of phytosphingosine and its acetylated derivatives increased to 142 mg/L, and also up to 650 mg/L under optimal conditions. However, no technology has been developed to mass-produce dihydrosphingosine, sphingosine, and glucosylceramide onto the cell surface in Y. lipolytica with high efficiency without acetylation.

### SUMMARY OF THE INVENTION

An object of the present disclosure is to provide a Y. lipolytica mutant strain with deletion of the SUR2 gene in a Y. lipolytica strain, and a method of secreting and producing, onto a cell surface, dihydrosphingosine, sphingosine, or glucosylceramide using the strain.

### Technical Solutions

To achieve the above object, the present disclosure provides a Y. lipolytica mutant strain with deletion of the SUR2 gene or suppressed expression thereof in a Y. lipolytica strain.

In addition, the present disclosure provides a method of producing dihydrosphingosine, sphingosine, or glucosylceramide, including culturing the Y. lipolytica mutant strain in a medium.

In addition, the present disclosure provides a Y. lipolytica mutant recombinant strain into which a gene encoding ceramidase is introduced in the Y. lipolytica mutant strain with enhanced secretion and production of dihydrosphingosine or sphingosine onto a cell surface, and provides a method of secreting and producing dihydrosphingosine or sphingosine onto the cell surface including culturing the Y. lipolytica mutant recombinant strain in a medium.

In addition, the present disclosure provides a Y. lipolytica mutant recombinant strain in which an SLD1 gene encoding Δ8 desaturase is additionally deleted from the Y. lipolytica mutant strain, with enhanced secretion and production of sphingosine or human glucosylceramide onto a cell surface, and provides a method of secreting and producing sphingosine or human glucosylceramide onto the cell surface including culturing the Y. lipolytica mutant recombinant strain in a medium.

The present disclosure relates to an oleaginous yeast Y. lipolytica sur2 mutant strain and a method of secreting and producing a sphingoid-based precursor, sphingosine, and sphingolipid onto a cell surface using the same, and to a technology to prepare a mutant strain of which productivity of dihydrosphingosine is remarkably enhanced by deleting a SUR2 gene that encodes dihydrosphingosine C4-hydroxylase in an oleaginous yeast Y. lipolytica and to secrete and produce dihydrosphingosine onto the cell surface without additional acetylation using the same. According to the present disclosure, using a Y. lipolytica sur2 mutant strain which is a GRAS that has been proven to be stable for the human body, it is possible to mass-produce dihydrosphingosine and also simplify an extraction process by secreting onto a cell surface by means of lipid droplets without acetylation unlike other yeasts. In addition, the Y. lipolytica sur2 mutant strain shows features that glucosylceramide, a sphingosine-based lipid, is also secreted on the cell surface with high efficiency. Therefore, it is expected that the Y. lipolytica sur2 mutant strain produced in the present disclosure may economically mass-produce dihydrosphingosine, which is a major substance commonly present in the production process of various sphingolipids, and furthermore, it may be provided as a parent strain for development of artificial yeasts that mass-produce various useful sphingosine-based substances such as various forms of sphingosines and ceramides through metabolic engineering.

### BRIEF DESCRIPTION OF DRAWINGS

FIG. 1 shows a diagram illustrating a sphingolipid biosynthesis pathway in mammals, plants, and yeasts.

FIG. 2 shows (A) a schematic diagram for production of a Y. lipolytica ku70 deficient strain and (B) a result of identifying KU70 gene disruption by PCR.

FIG. 3A shows analysis of amino acid sequences and domains of S. cerevisiae, P. ciferrii, and Y. lipolytica Sur2 proteins, and FIG. 3B shows a schematic diagram of a cassette vector for production of a sur2 deficient strain and a result of identifying the SUR2 gene disruption by PCR.

FIG. 4A shows growth curves depending on culture medium composition of Y. lipolytica sur2 deficient strains, FIG. 4B shows micrographs of fungus body, and FIG. 4C shows results of analyzing growth under various cell wall weakening conditions and osmotic stress conditions.

FIG. 5A shows a structure of dihydrosphingosine, FIG. 5B shows a copper sulfate (CuSO₄) detection method of cell surface, cell culture, and cytosol samples to detect dihydrosphingosine produced and secreted by Y. lipolytica sur2 deficient strains, FIG. 5C shows a TLC result analyzed by ninhydrin detection, and FIG. 5D shows a TLC analysis result in which dihydrosphingosine secretion and production efficiency of Y. lipolytica sur2 deficient strains is compared with that of S. cerevisiae sur2 deficient strains and a graph of quantification thereof.

FIG. 6A shows results of HPLC analysis for dihydrosphingosine extracted from cell surfaces of Y. lipolytica sur2 deficient strains, and FIGS. 6B and 6C show results of LC/MS/MS analysis for dihydrosphingosine extracted from cell surfaces of Y. lipolytica sur2 deficient strains.

FIG. 7A shows TLC analysis results of glucosylceramide extracted from the cell surface, cell culture media, and cytosol of Y. lipolytica sur2 deficient strains, FIG. 7B shows an LC/MS analysis result for cell surface samples, and FIG. 7C shows TLC analysis results of identifying changes in inositol sphingolipid extracted from the cytosol.

FIG. 8A shows a diagram of a sphingolipid biosynthesis pathway of a wild-type Y. lipolytica strain, and FIG. 8B shows a diagram of a sphingolipid biosynthesis pathway of a sur2 deficient strain.

FIG. 9A shows a strategy of amplifying sphingosine production by introducing various forms of ceramidase genes into a Y. lipolytica sur2 deficient strain, and FIGS. 9B and 9C show TLC analysis results.

FIG. 10A shows analysis of amino acid sequences and domains of Y. lipolytica, C. albicans, and P. pastoris Sld1 proteins, FIG. 10B shows schematic diagrams of a cassette vector for production of a sld1 deficient strain and results of identifying SLD1 gene disruption by PCR in Y. lipolytica, and FIG. 10C shows results of analyzing growth of Y. lipolytica sld1 deficient strain under various cell wall weakening conditions and osmotic stress conditions.

FIG. 11A shows a diagram illustrating a sphingolipid biosynthesis pathway of a Y. lipolytica sld1 deficient strain, FIG. 11B shows a TLC analysis result of comparing efficiency in secretion and production of sphingolipid extracted from the cell surface, FIG. 11C shows TLC analysis results of glucosylceramide, and FIG. 11D shows an LC/MS analysis result.

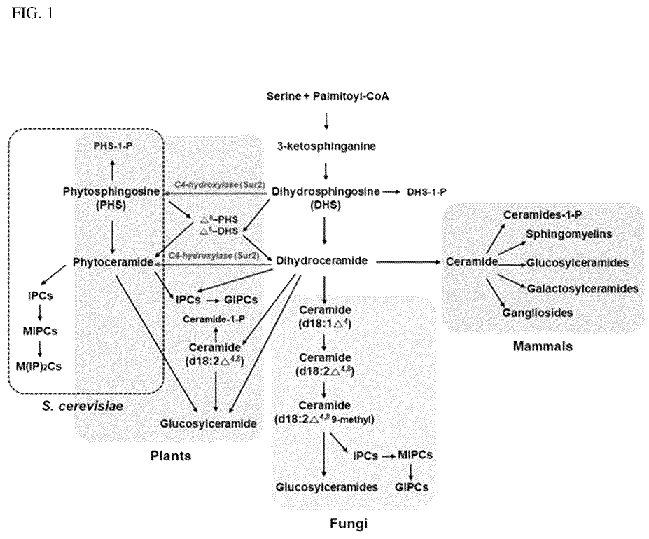

### DETAILED DESCRIPTION OF THE INVENTION

Therefore, the present inventors completed the present disclosure by preparing a mutant strain with deletion of a SUR2 gene to block a biosynthesis pathway for phytosphingosine and phytoceramide in oleaginous yeast Y. lipolytica and established a technique in which dihydrosphingosine, a sphingosine precursor, and glucosylceramide, a sphingosine-based lipid, are secreted and produced onto a cell surface with high efficiency using the mutant strain.

The present disclosure provides a Y. lipolytica mutant strain with deletion of the SUR2 gene or suppressed expression thereof in a Y. lipolytica strain.

The SUR2 gene is a gene encoding a dihydrosphingosine C4-hydroxylase and may be represented by SEQ ID NO: 4, but is not limited thereto. On the other hand, an amino acid sequence of the Sur2 protein may be represented by SEQ ID NO: 3.

More preferably, the Y. lipolytica mutant strain may be a strain (Yarrowia lipolytica sur24) deposited under Accession number KCTC 14592BP.

More preferably, the Y. lipolytica mutant strain may have enhanced secretion and production of dihydrosphingosine or glucosylceramide onto a cell surface.

In addition, the present disclosure provides a method of producing dihydrosphingosine or glucosylceramide, including culturing the Y. lipolytica mutant strain in a medium.

Preferably, the medium may include, but is not limited to, glycerol as a carbon source.

Preferably, the method allows dihydrosphingosine or glucosylceramide to be secreted onto the cell surface by means of lipid droplets without additional acetylation process.

In addition, the present disclosure provides a Y. lipolytica mutant recombinant strain in which a gene encoding ceramidase is introduced into the Y. lipolytica mutant strain, with enhanced secretion and production of dihydrosphingosine or sphingosine onto a cell surface.

Preferably, the gene encoding ceramidase may be a human alkaline ceramidase 1 gene (hACER1) represented by SEQ ID NO: 5, a human alkaline ceramidase 2 gene (hACER2) represented by SEQ ID NO: 6, a human alkaline ceramidase 3 gene (hACER3) represented by SEQ ID NO: 7, a mouse alkaline ceramidase 1 gene (mACER1) represented by SEQ ID NO: 8, an N-fragment deficient human alkaline ceramidase 2 gene (hACER2(N12Δ)) represented by SEQ ID NO: 9, or a Y. lipolytica ceramidase gene (YIYDC1) represented by SEQ ID NO: 10, but is not limited thereto.

Amino acid sequences of the human-derived alkaline ceramides hACER1, hACER2, and hACER3, as used herein, may be NCBI accession no. NP_597999.1, NCBI accession no. NP_001010887.2, and NCBI accession no. AAH73853.1, respectively, an amino acid sequence of mouse-derived ceramidase mACER1 may be NCBI accession no. NP_783858.1, and an amino acid sequence of Y. lipolytica-derived ceramidase YIYdc1 may be NCBI accession no. QNP97986.1, but are not limited thereto.

In addition, the present disclosure provides a method of secreting and producing dihydrosphingosine or sphingosine onto a cell surface, including culturing, in a medium, a Y. lipolytica mutant recombinant strain in which a gene encoding ceramidase is introduced into the Y. lipolytica mutant strain.

In addition, the present disclosure provides a Y. lipolytica mutant recombinant strain in which an SLD1 gene encoding Δ8 desaturase is additionally deleted from a Y. lipolytica mutant strain, with enhanced secretion and production of sphingosine or human glucosylceramide onto the cell surface.

Preferably, the SLD1 gene encoding Δ8 desaturase may be represented by SEQ ID NO: 12, but is not limited thereto. On the other hand, an amino acid sequence of an SLD1 protein may be represented by SEQ ID NO: 11.

Preferably, the human glucosylceramide may be, but is not limited to, glucosylceramide GlcCer(d18:1(4E)/16:0(2OH)).

More preferably, the Y. lipolytica mutant recombinant strain may be a strain (Yarrowia lipolytica sld14 sur24) deposited under Accession number KCTC 14980BP.

In addition, the present disclosure provides a method of secreting and producing sphingosine or human glucosylceramide onto a cell surface, including culturing, in a medium, a Y. lipolytica mutant recombinant strain in which an SLD1 gene encoding Δ8 desaturase is additionally deleted from the Y. lipolytica mutant strain.

Furthermore, using the Y. lipolytica sur2 deficient strain or sld1 sur2 double deficient mutant strain prepared in the present disclosure as a parent strain, it is possible to prepare strains that mass-produce various forms of useful sphingosine-based substances through metabolic engineering.

Hereinafter, example embodiments will be described in detail to help the understanding of the present disclosure. However, the following example embodiments are merely illustrative of the content of the present disclosure, and the scope of the present disclosure is not limited by the following example embodiments. The example embodiments of the present disclosure are provided to more completely explain the present disclosure to those skilled in the art.

### <Example 1> Preparation of Yarrowia lipolytica Sur2 Deficient Strain

1-1. Preparation of Yarrowia lipolytica Ku70 Strain

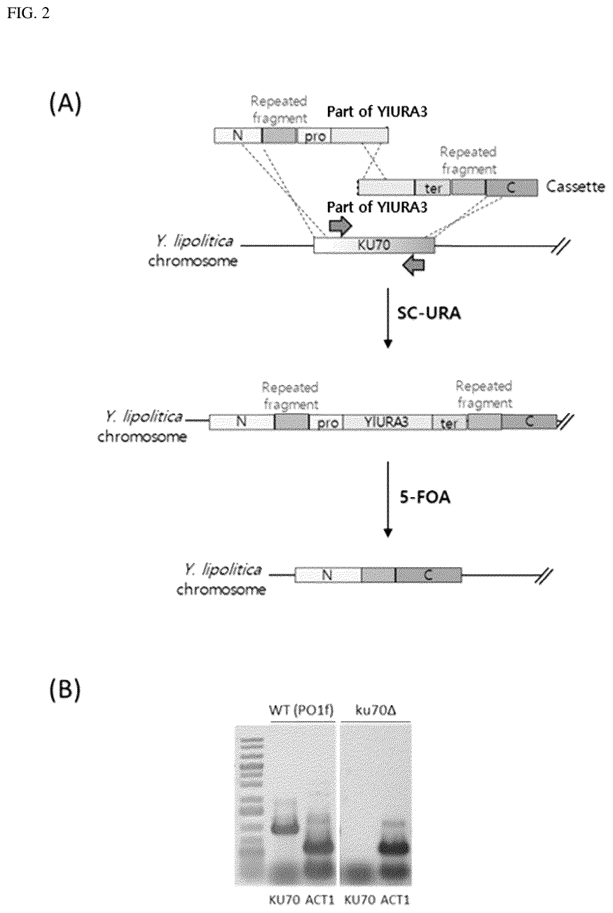

In order to increase an efficiency of homologous recombination (HR)-based genetic manipulation in the Y. lipolytica strain, a mutant strain from which non-homologous end joining (NHEJ) is removed through KU70 gene disruption was produced first. Ku70 and Ku80 together form a Ku heterodimer and bind to the DNA double-stranded cleaved end, which is involved in the non-homologous end joining pathway of DNA repair. With the genome of the Y. lipolytica PO1f (MATA ura3-302 leu2-270 xpr2-322 axp2-deltaNU49 XPR2::SUC2) strain as a template, 5′ portion of the KU70 gene was amplified by PCR using Ylku70dNfw and Ylku70dNrv primers listed in Table 1 as well as a promoter −286 bp site and the open reading frame (ORF) 472 bp of the KU70 gene YALI0C08701g gene (SEQ ID NO: 2), the 3′ portion of the KU70 gene was amplified by PCR using Ylku70dCfw and Ylku70dCrv as well as KU70 orf 1555 bp site fw and terminator 480 bp, and the N and C fragments were ligated through additional fusion PCR and introduced into pT-blunt vector to obtain a pTB-ku70dNC vector. Subsequently, the YIURA3 gene with the same specific sequence of 580 bp back and forth was introduced into the MluI site of the pTB-ku70dNC vector to obtain a pTB-ku70dNC-YIURA3 vector. Using pTB-ku70dNC-YIURA3 as a template, the N fragment was obtained using Ylku70dNfw and YIURA3Nrv primers listed in Table 1, the C fragment was obtained using YIURA3Cfw and Ylku70dCrv primers, and the fragments were transduced into the Y. lipolytica PO1f strain to prepare a Ylku70 disrupted strain through homologous recombination in cells (FIG. 2A). The exact disruption of the KU70 gene was detected by PCR after chromosome extraction (FIG. 2B). Subsequently, the Ylku70 deficient strain was cultured in YPD media including 1 mg/ml 5-FOA to recover the URA3 marker that was used for KU70 deletion, and the URA3 gene was induced to be removed (FIG. 2A).

1-2. Preparation of Sur2 Deficient Strain Using Yarrowia lipolytica Ku70 Strain

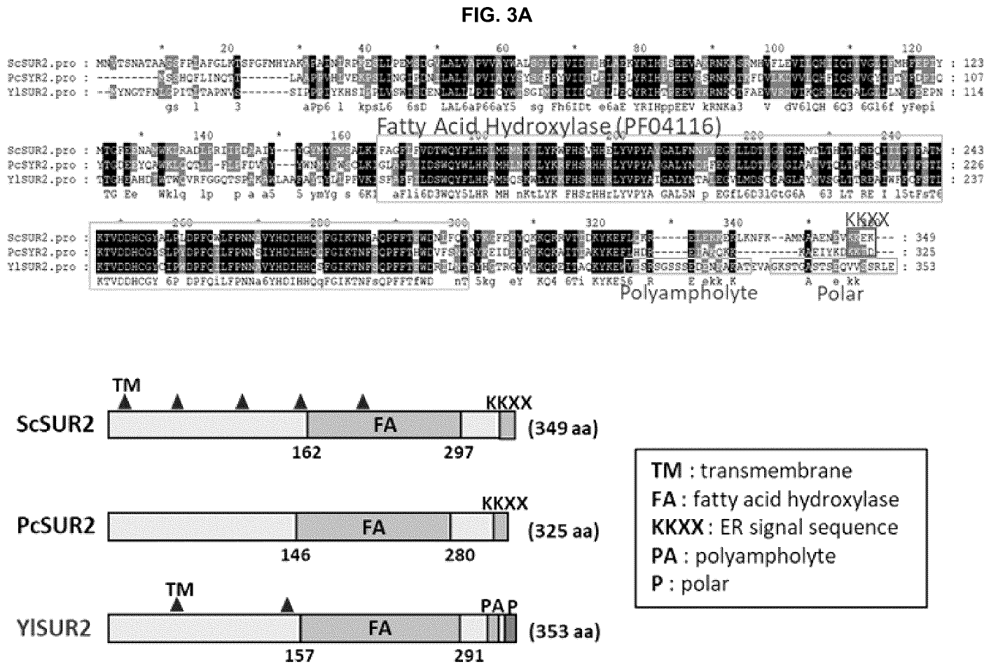

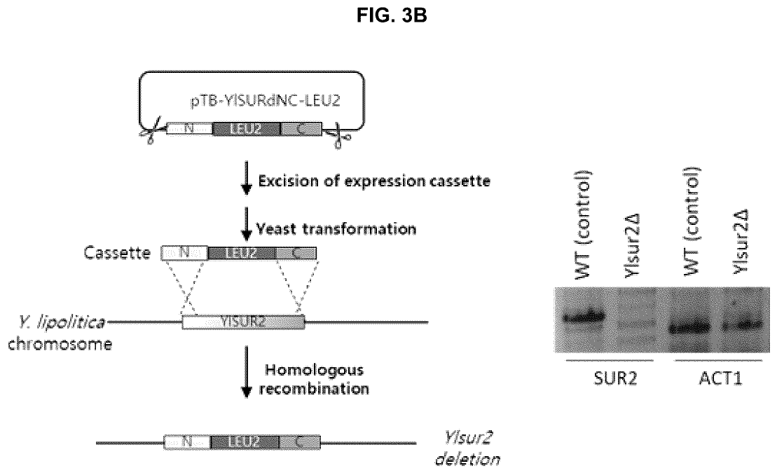

The inventors sought to discover a gene (SEQ ID NO: 4) encoding a protein that is 45.6% identical to a S. cerevisiae Sur2 protein in order to block a phytosphingosine biosynthesis pathway in Y. lipolytica, and to disrupt using homologous gene recombination. The Y. lipolytica Sur2 protein showed some structural differences from other yeasts. It had slightly fewer membranous domains than S. cerevisiae and slightly more membranous domains than P. ciferrii, and instead of having KKXX, an ER signal sequence at the C terminus, it had a polyampholyte and polar structure. However, the fatty acid hydroxylase domain was well conserved (FIG. 3A).

To prepare a Y. lipolytica SUR2 gene disruption cassette, after amplifying 5′ portion of the SUR2 gene by PCR using Ylsur2dNfw and Ylsur2dNrv primers listed in Table 1 as well as the SUR2 promoter −668 bp site and the open reading frame (ORF) 100 bp and amplifying 3′ portion of the SUR2 gene by PCR using Ylsur2dCfw and Ylsur2dCrv as well as SUR2 orf 1,011 bp site fw and terminator 639 bp, and then ligating the 5′ and 3′ portions through fusion-PCR using the MluI-NheI-SalI portion possessed by both the 5′ and 3′ portions, the pTB-SUR2dNC vector was prepared by introducing into a pT blunt vector. Afterwards, the YILEU2 gene fragment was amplified by PCR and treated with NheI/SalI to ligate with the NheI/SalI-treated pTB-SUR2dNC vector and produce a pTB-SUR2d NC-YILEU2 vector. Afterwards, the cassette was prepared by treating the pTB-SUR2dNC-YILEU2 vector with SpeI/NsiI, and then the Y. lipolytica ku70 deficient strain prepared was transformed to produce the Y. lipolytica sur2 deficient strain (FIG. 3B).

The primers used in the present disclosure are listed in Table 1.

TABLE 1

| | | Descrip- |
|---|---|---|
| Primer | Sequence (5′→3′) | tion |
| ku70dNfw | gagcggtttgctcgaaaccagt | YlKU70 |
| | (SEQ ID NO: 13) | dele- |
| ku70dNrv | tacagtcgacgcgtgggcgt | tion |
| | atcgattatcgtcgtctgtgat | |
| | atagatgattcg | |
| | (SEQ ID NO: 14) | |
| ku70dCfw | cgatacgcccacgcgtcgactg | |
| | taaggctgagaagaaagtcaag | |
| | ag | |
| | (SEQ ID NO: 15) | |
| ku70dCrv | gagcggtttgctcgaaaccag | |
| | (SEQ ID NO: 16) | |
| URA3Nrv | cagctcggccagcatgag | |
| | (SEQ ID NO: 17) | |
| URA3Cfw | ccaacctgtgtgcttctctgg | |
| | (SEQ ID NO: 18) | |
| Ylsur2dNfw | cgaccgaaaatgtgcaagcg | YlSUR2 |
| | (SEQ ID NO: 19) | dele- |
| Ylsur2dNrv | acgagctagccgcgcgacgcgt | tion |
| | cgagtggtggaatggagtgttt | |
| | g | |
| | (SEQ ID NO: 20) | |
| Ylsur2dCfw | gaacagtcgactggagcttcta | |
| | cttccgagc | |
| | (SEQ ID NO: 21) | |
| Ylsur2dCrv | catgcgtcctctcctccacgat | |
| | tc | |
| | (SEQ ID NO: 22) | |
| LEU2fw | cgcgcggctagctcgtcgcctg | |
| | agtcatcatttat | |
| | (SEQ ID NO: 23) | |
| LEU2rv | gctccagtcgactgttcggaaa | |
| | tcaacggatgctc | |
| | (SEQ ID NO: 24) | |
| Scsur2dNfw | tcaccccgcgtccttcttcggg | ScSUR2 |
| | cagtcgcgcttttctccggctt | dele- |
| | ctgcggaaattgaagctctaat | tion |
| | ttgtgagtttag | |
| | (SEQ ID NO: 25) | |
| Scsur2dCrv | ctcatcagttttcgaagatttc | |
| | aagaaagtccgaagaacattta | |
| | agaccttttcaattcaattcat | |
| | cattttttttttattctt | |
| | (SEQ ID NO: 26) | |
| TEF1pfw | ccggaattcagagaccgggttg | hACER1 |
| | gcggcgt | expres- |
| | (SEQ ID NO: 27) | sion |
| TEF1p(h1) | tgcgaagatcgaaggcattttg | |
| rv | aatgattcttatactcagaagg | |
| | aaatgc | |
| | (SEQ ID NO: 28) | |
| hACER1fw | gagtataagaatcattcaaaat | |
| | gccttcgatcttcgcataccaa | |
| | tc | |
| | (SEQ ID NO: 29) | |
| hACER1rv | atcaccggcaaactatctgtct | |
| | aacaatccttgtcatctccccg | |
| | aa | |
| | (SEQ ID NO: 30) | |
| XPR2t(h1) | ggggagatgacaaggattgtta | |
| fw | gacagatagtttgccggtgata | |
| | attctctta | |
| | (SEQ ID NO: 31) | |
| XPR2trv | ctagtctagaggggggctagcc | |
| | acgggcatctcacttgcgc | |
| | (SEQ ID NO: 32) | |
| TEF1p(h2) | caccaatgaggtgctcccattt | hACER2 |
| rv | tgaatgattcttatactcagaa | expres- |
| | ggaaatgc | sion |
| | (SEQ ID NO: 33) | |
| hACER2fw | gagtataagaatcattcaaaat | |
| | gggagcacctcattggtgg | |
| | (SEQ ID NO: 34) | |
| hACER2rv | atcaccggcaaactatctgtct | |
| | aggtaatcttaacgctgctctt | |
| | tttgttag | |
| | (SEQ ID NO: 35) | |
| XPR2t(h2) | agagcagcgttaagattaccta | |
| fw | gacagatagtttgccggtgata | |
| | attctctta | |
| | (SEQ ID NO: 36) | |
| TEF1p(h2Δ) | cagtccacctcagacgacattt | hACER2 |
| rv | tgaatgattcttatactcagaa | (12Δ) |
| | ggaaatgc | expres- |
| | (SEQ ID NO: 37) | sion |
| hACER2 | gagtataagaatcattcaaaat | |
| (12Δ)fw | gtcgtctgaggtggactgg | |
| | (SEQ ID NO: 38) | |
| hACER2 | caccggcaaactatctgtctag | |
| (12Δ)rv | gtaatcttaacgctgctctttt | |
| | tg | |
| | (SEQ ID NO: 39) | |
| XPR2t(h2Δ) | gagcagcgttaagattacctag | |
| fw | acagatagtttgccggtgata | |
| | attctctta | |
| | (SEQ ID NO: 40) | |
| TEF1p(h3) | cgatctgcggcgggggccattt | hACER3 |
| rv | tgaatgattcttatactcagaa | expres- |
| | ggaaatgc | sion |
| | (SEQ ID NO: 41) | |
| hACER3fw | gagtataagaatcattcaaaat | |
| | ggcccccgccgcagatcg | |
| | (SEQ ID NO: 42) | |
| hACER3rv | atcaccggcaaactatctgtct | |
| | agtgttttcgaagaggttcaaa | |
| | gagaatc | |
| | (SEQ ID NO: 43) | |
| XPR2t(h3) | ctttgaacctcttcgaaaacac | |
| fw | tagacagatagtttgccggtga | |
| | taattctctta | |
| | (SEQ ID NO: 44) | |
| TEF1p(m1) | tccagggacgtgcattttgaat | mACER1 |
| rv | gattcttatactcagaaggaaa | expres- |
| | tgc | sion |
| | (SEQ ID NO: 45) | |
| mACER1fw | gagtataagaatcattcaaaat | |
| | gcacgtccctggaacgag | |
| | (SEQ ID NO: 46) | |
| mACER1rv | atcaccggcaaactatctgtct | |
| | aacagttcttatcattttcctg | |
| | gatctc | |
| | (SEQ ID NO: 47) | |
| XPR2t(m1) | ggaaaatgataagaactgttag | |
| fw | acagatagtttgccggtgataa | |
| | ttctctta | |
| | (SEQ ID NO: 48) | |
| TEF1p | gggattcctccaggtagcattt | YIYDC1 |
| (YDC1)rv | tgaatgattcttatactcagaa | expres- |
| | ggaaatgcttaacg | sion |
| | (SEQ ID NO: 49) | |
| YIYDC1fw | gagtataagaatcattcaaaat | |
| | gctacctggaggaatcccatac | |
| | c | |
| | (SEQ ID NO: 50) | |
| YIYDC1rv | atcaccggcaaactatctgtct | |
| | agttgtgatggttaacaga | |
| | (SEQ ID NO: 51) | |
| XPR2t | ctgttaaccatcacaactagac | |
| (YDC1)fw | agatagtttgccggtgataatt | |
| | ctcttaac | |
| | (SEQ ID NO: 52) | |
| YISLD1dNfw | atgcagacggataggtggtggg | YISLD1 |
| | (SEQ ID NO: 53) | dele- |
| YISLD1dNrv | ctcaagctggacaactggctcg | tion |
| | atggtagctacttacgcgatcg | |
| | tagggccagctg | |
| | (SEQ ID NO: 54) | |
| YISLD1dCfw | ccatcgatgaatgcgctagcat | |
| | cccggtcgacgtgactgtgagc | |
| | gacatcttcg | |
| | (SEQ ID NO: 55) | |
| YISLD1dCrv | cggctaaccagaaacggtccta | |
| | tc | |
| | (SEQ ID NO: 56) | |

*Underlined: restriction enzyme

The strains produced in the present disclosure are listed in Table 2.

TABLE 2

| | | Source/ |
|---|---|---|
| Strain | Description | Reference |
| Y. lipolytica PO1f | MATA ura3-302 leu2-270 | ATCC |
| | xpr2-322 axp2- | MYA-2613 |
| | deltaNU49 XPR2::SUC2 | |
| Ylku70 Δ | MATA ura3-302 leu2-270 | This study |
| | xpr2-322 axp2-deltaNU49 | |
| | XPR2::SUC2 ku70:tc | |
| S. cerevisiae BY4742 | MAT a his3A1 leu2A0 | Open |
| | lys2A0 ura3A0 | Biosystems |
| Ylsur2 Δ | MATA ura3-302 leu2-270 | This study |
| | xpr2-322 axp2-deltaNU49 | |
| | XPR2::SUC2 ku70::tc | |
| | sur 2::YlLEU2 | |
| Scsur2 Δ | MATα his3A1 leu2A0 | This study |
| | lys2A0 ura3A0 | |
| | sur2::ScURA3 | |
| hACER1/Ylsur2 Δ | pIMR53-hACER1/Ylsur2Δ | This study |
| hACER2/Ylsur2 Δ | pIMR53-hACER2/Ylsur2Δ | This study |
| hACER2(12 Δ)/Ylsur2 Δ | pIMR53-hACER2(12Δ)/Ylsur2Δ | This study |
| hACER3/Ylsur2 Δ | pIMR53-hACER3/Ylsur2Δ | This study |
| mACER1/Ylsur2 Δ | pIMR53-mACER1/Ylsur2Δ | This study |
| YlYDC1/Ylsur2 Δ | pIMR53-YlYDC1/Ylsur2Δ | This study |
| Ylsld1 Δ | MATA ura3-302 leu2-270 | This study |
| | xpr2-322 axp2-deltaNU49 | |
| | XPR2::SUC2 ku70::tc | |
| | sld1::YIURA3 | |
| Ylsld1 Δ sur2 Δ | MATA ura3-302 leu2-270 | This study |
| | xpr2-322 axp2-deltaNU49 | |
| | XPR2::SUC2 ku70::tc | |
| | sur 2::YlLEU2 sld1::YlURA3 | |

### <Example 2> Analysis of Growth Features of Yarrowia lipolytica Sur2 Deficient Strain

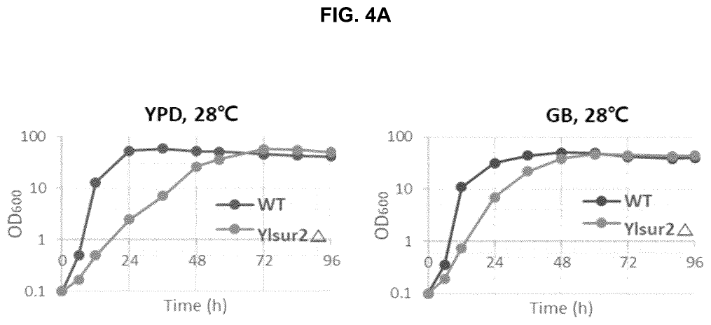

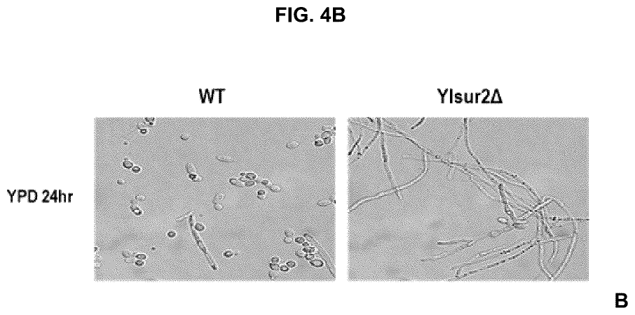

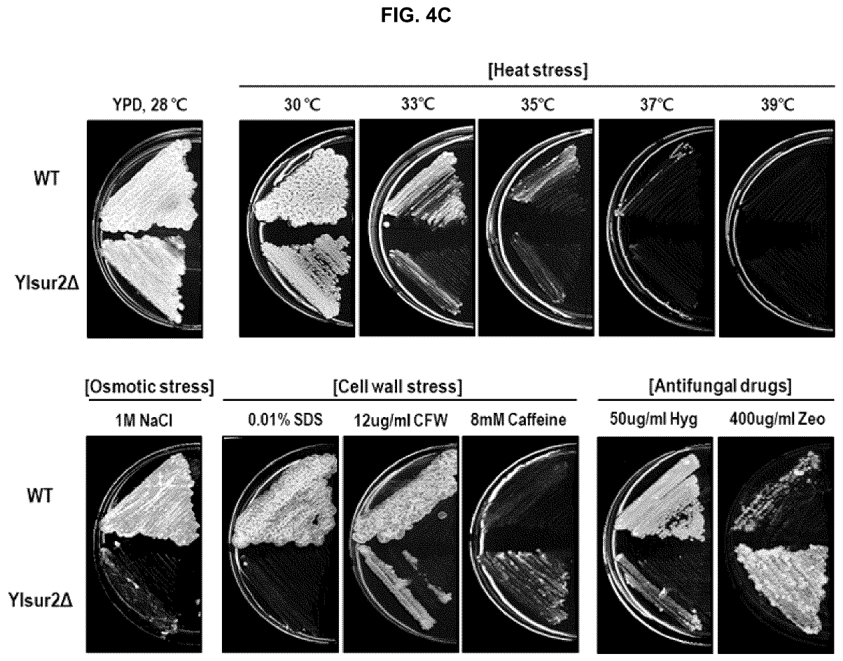

In order to identify the growth pattern according to the carbon source type and medium composition of the Y. lipolytica sur2 deficient strain (Ylsur24) produced in Example 1, comparative analysis was conducted with Ylku704/PO1f, a parent strain of Ylsur24. The strain culture medium pre-cultured in 2 ml of YPD medium was inoculated with OD₆₀₀=0.1 in 25 ml of YPD or GB medium and cultured in a mixed culture machine at 28° C. and 220 rpm, and the growth curve graph was obtained by measuring the OD₆₀₀ value at regular intervals. As a result, compared to the WT strain, which showed the highest value of OD₆₀₀ at about 36 hours of YPD culture, the Ylsur2 deficient strain showed the highest OD₆₀₀ value at 72 hour, which is about twice as long. In other words, it was determined that the growth pattern of the Ylsur2 deficient strain had a long lag phase and logarithmic growth phase, thereby taking a relatively long time to reach the stationary phase. In addition, it was found that when cultured in the GB medium including 8% glycerol rather than glucose as a carbon source, the time to reach the plateau was shortened, overcoming the growth difference with wild-type strains to some extent (FIG. 4A).

In addition, micrographs obtained by culturing in YPD medium for 24 hours showed that the Ylsur2 deficient strain grew in the hyphae form at a much higher proportion than the parent strain (FIG. 4B).

In order to investigate the weak portions of the intracellular structure of the Y lipolytica sur2 deficient strain, the growth patterns were identified under various YPD medium conditions including inducers of temperature stress)(30˜39° C.), cell wall and osmotic stress (NaCl, SDS, CFW (=Fluorescent Brightener 28), caffeine), and antibiotics. As a result of smearing onto the YPD plate and grown for 4 days, it was found that the Ylsur2 deficient strain seemed to become very sensitive to temperature, as its growth was stunted from 30° C. and barely grew above 33° C. In addition, it was found that it became highly sensitive to osmotic pressure and cell wall stress, and growth was reduced even under medium conditions including hygromycin B, a protein synthesis inhibitor. This is thought to be the result of the weakening of cell membranes due to abnormal production of sphingolipids, making the cell wall vulnerable to various stresses and also having antibiotic substances easily penetrate into the cell. However, resistance was also shown in some media including caffeine, one of the cell wall stress conditions, and zeocin, an inducer of DNA double-strand separation (FIG. 4C).

### <Example 3> Identification of Secretion and Production of Dihydrosphingosine of Y. lipolytica Sur2 Deficient Strain onto Cell Surface by TLC Analysis

Due to hydrophobicity, sphingolipid components are clustered on the surface of the cell rather than being secreted out of the medium. Therefore, in order to collect the sphingolipids clustered on the cell surface, cell pellets were collected after culture, 10 ml of methanol was added per 1 g, and sonication was performed for 30 minutes. Afterwards, the sample dissolved in 10 ml of methanol was concentrated 10 times to 1 ml through drying, and the final cell surface sample was obtained. For the cell culture medium (supernatant) sample, the supernatant separated by centrifugation of the strain culture medium was mixed with the same amount of chloroform:methanol (2:1, v/v), and the centrifuged lower layer was used. In the case of cytosol samples, the remaining cell pellets were released in chloroform:methanol:water (20:10:1, v/v) after collecting the cell surface sample, and the cells were disrupted with a beadbeater, lyophilized, and dissolved in methanol. All samples were prepared by drying in the same way and concentrating with 1 ml of methanol.

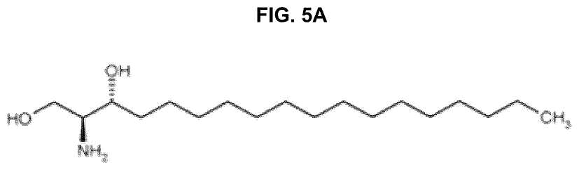

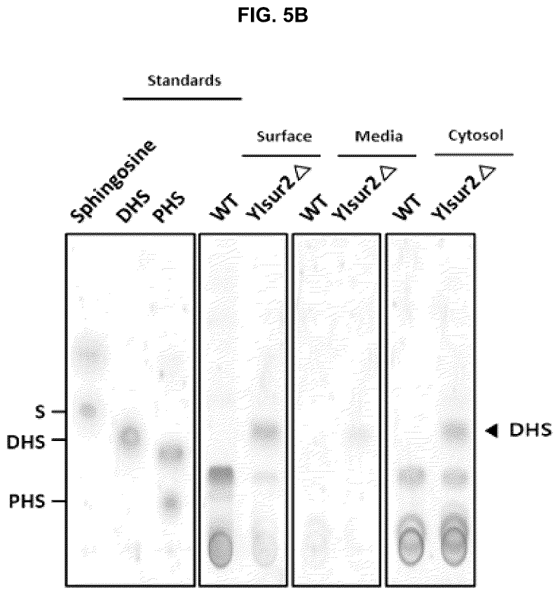

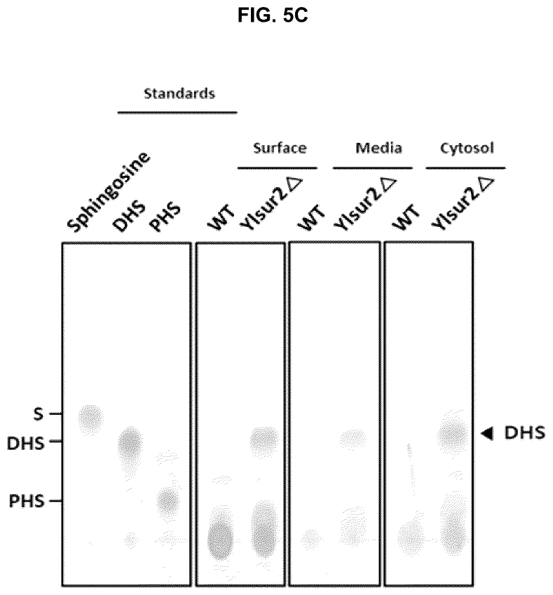

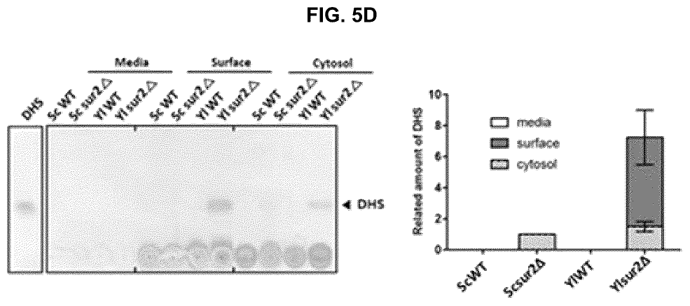

Samples extracted from TLC silica gel 60 plates were dropped by 10 ul each and developed using a non-polar solvent consisting of chloroform:methanol:ammonia water (18:2:1). After development, the copper sulfate (CuSO4) detection method was used to analyze the overall lipid changes, and the ninhydrin solution was used to identify the sphingoid bases, among which dihydrosphingosine (FIG. 5A). After development, the completely dried TLC plate was soaked in a solution of copper sulfate and ninhydrin and then completely dried and burned at 200° C. for detection. The detection solution used was dissolved in 8% sulfuric acid in case of 10% copper sulfate, and 0.2% ninhydrin was dissolved in ethanol. For comparison, it was developed together with sphingosine, dihydrosphingosine, and phytosphingosine standard products.

As a result of TLC analysis using copper sulfate detection method, it was possible to detect dihydrosphingosine in all cell surface, culture supernatant, and cytosol samples of Ylsur2 deficient strains. This may be interpreted as the accumulation of dihydrosphingosine due to deletion in the SUR2 gene, which is responsible for phytosphingosine synthesis, and the fact that dihydrosphingosine is detected not only in cytosol but also in cell surface and culture supernatant samples indicates that it may be secreted by means of lipid droplets (FIG. 5B).

In this experiment, in order to identify only the pure sphingoid base, also used was a ninhydrin detection method that may react with the amino group (NH₂) on serine, which is the basic backbone of sphingolipids. Through the result that the dihydrosphingosine band identified by the copper sulfate detection method was also determined by the ninhydrin detection method, it was redetermined that the substance is the spingoid base itself, that is, dihydrosphingosine with no substance attached to the amino group (—NH₂) of sphingolipid (FIG. 5C). From these results, it may be inferred that dihydrosphingosine accumulates in the Y. lipolytica sur2 deficient strain, and a large amount thereof is secreted to the cell surface and medium.

In order to determine that the secretion and production of dihydrosphingosine in Y. lipolytica sur2 deficient strains is a unique phenomenon of this strain unlike other yeasts, the efficiencies in secretion of dihydrosphingosine onto cell surfaces of Saccharomyces cerevisiae and Y. lipolytica were compared and analyzed. The sample extraction method and TLC analysis method are the same as in Example 3, and ninhydrin solution was used for detection. As a result of TLC analysis, no dihydrosphingosine was found on the cell surface and culture supernatant of the S. cerevisiae sur2 deficient strain, indicating that dihydrosphingosine is secreted and produced specifically in the Y. lipolytica sur2 deficient strain (FIG. 5D).

### <Example 4> Determination of Secretion and Production of Dihydrosphingosine in Y. lipolytica Sur2 Deficient Strain Through HPLC and LC/MS/MS Analysis

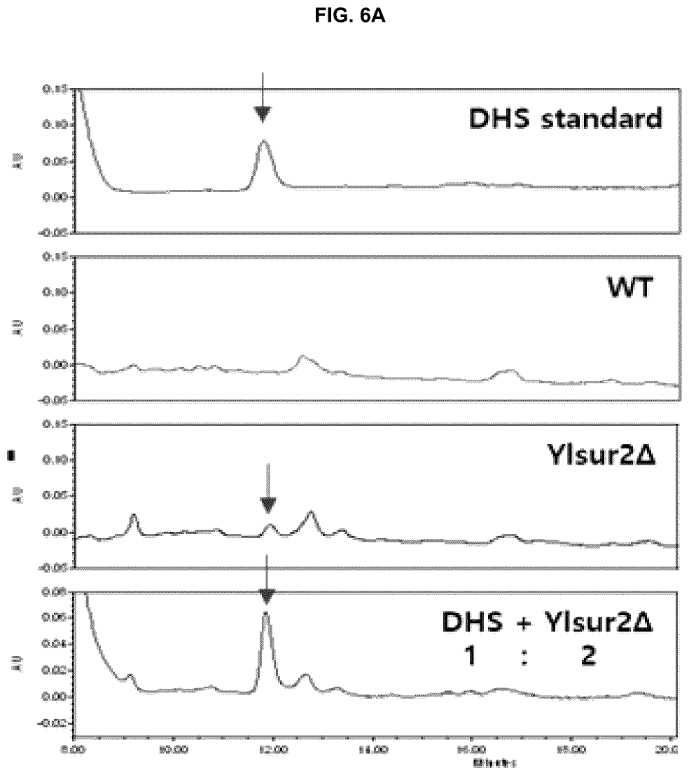

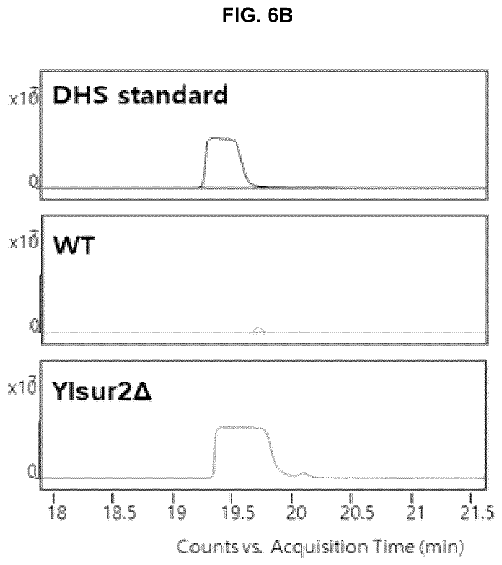

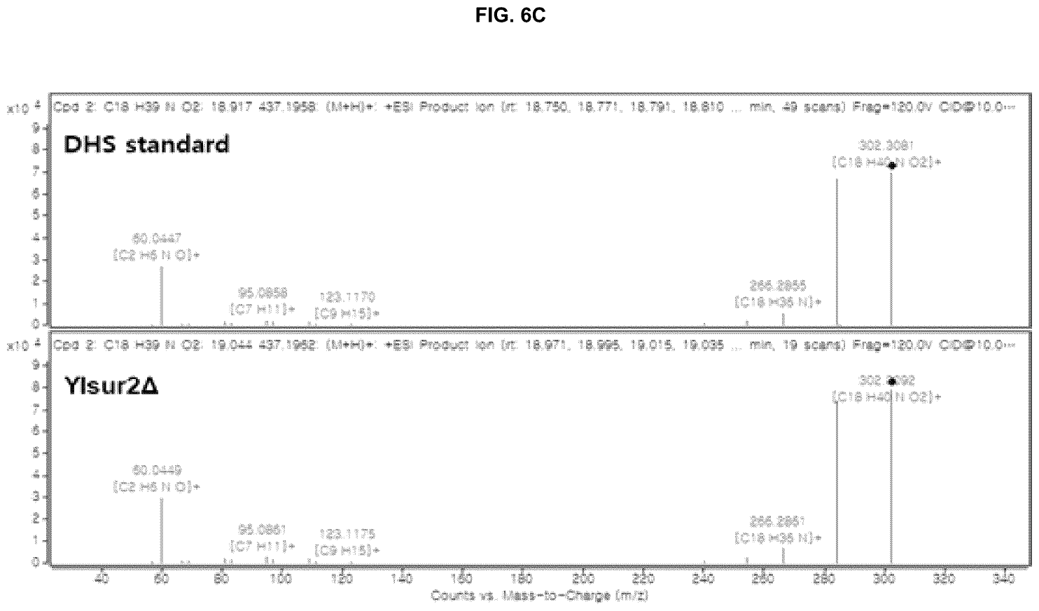

HPLC analysis was performed using the cell surface sphingolipid sample used in Example 3. As a result of analyzing the Cosmosil packed column 5C18-PAQ, 4.1ID x 250 mm column (40° C.) and MeOH:water:ammonia (80:19.5:0.5) mobile phase at a rate of 0.5 ml/min with a 210 mM UV detector, the bands were detected in the Y. lipolytica sur2 deficient strain at the same location as the dihydrosphingosine standard product (FIG. 6A). On the other hand, the results of LC/MS/MS analysis also showed that the substance produced in the Y. lipolytica sur2 deficient strain is identical to the dihydrosphingosine standard product (FIG. 6B and FIG. 6C).

Therefore, the dihydrosphingosine-secreting and producing yeast strain developed in the present disclosure is expected to be used as a host for the cell surface mass production of sphingolipids, which is emerging as an effective alternative to complex intracellular extraction methods in terms of productivity and simplicity in separation and purification.

### <Example 5> Identification of Glucosylceramide and Inositol Sphingolipid Biosynthesis in Y. lipolytica Sur2 Deficient Strain by TLC Analysis

In order to analyze the overall changed lipid components in the Y. lipolytica sur2 deficient strain, it was intended to identify glucosylceramide and inositol sphingolipid, which are substances at the final stage of the sphingolipid synthesis pathway. Sample preparation was the same as in Example 3, and TLC analysis was performed. In order to detect glucosylceramide, the component of the development solvent was changed, development was performed with chloroform:methanol:acetic acid:water (20:3.5:2.5:0.7, v/v), and after moistening in the detection solution, drying was performed completely, followed by a reaction at 80° ° C. The detection solution is dissolved in 0.1 g of orcinol per 45 ml of sulfuric acid:water:ethanol (5:13:27), which is one of the detection methods used to identify glycosides and glycolipids in TLC analysis, and it reacts with glucose in glucosylceramide and appears as a purple band on the TLC plate. For comparison, GlcCer(d18:1(4E)/16:0) and GlcCer (d18:2(4E,8E)/16:0(2OH)) were used for the standard products.

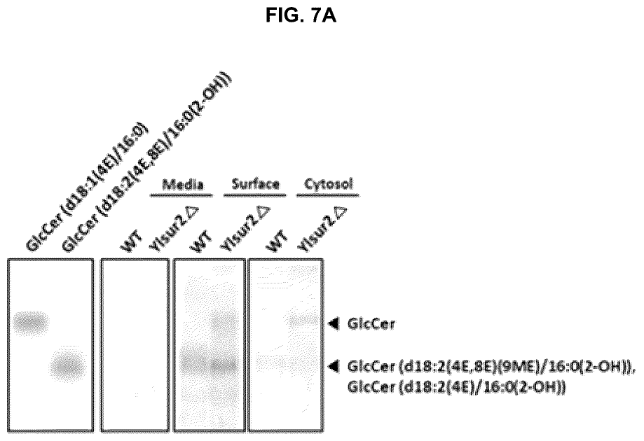

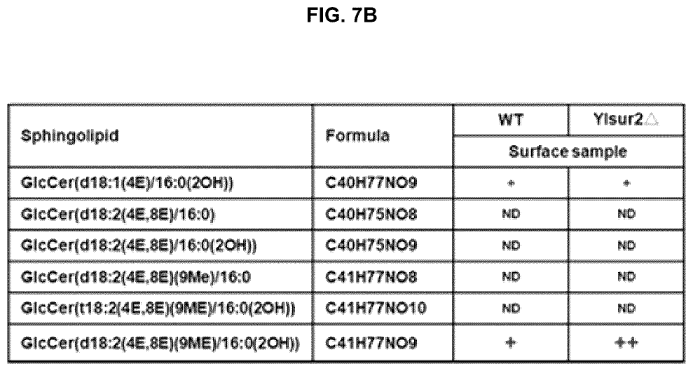

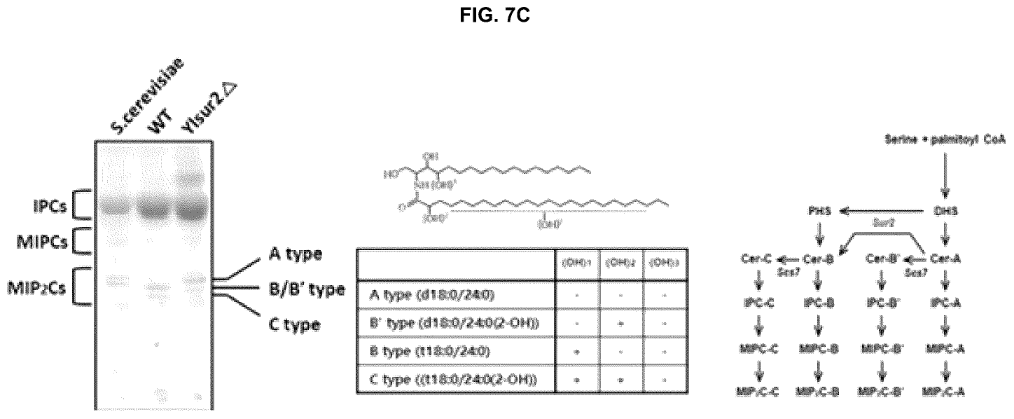

As a result of TLC analysis, a band consistent with the GlcCer(d18:2(4E,8E)/16:0(2OH)) standard product appeared in the cell surface samples of the wild-type Y. lipolytica strain and the sur2 deficient strain, and the bands were slightly darkened in the Ylsur2 deficient strain. In addition, bands were shown only in the Ylsur2 deficient strain at a position similar to that of the GlcCer (d18:1(4E)/16:0) standard product (FIG. 7A).

As a result of performing LC/MS analysis using the cell surface samples with the amount highest of glucosylceramide identified, highest amount of GlcCer(d18:2(4E,8E)(9Me)/16:0(2OH)) was present in both the wild-type strain and the Y. lipolytica sur2 deficient strain, with approximately a threefold increase in the Ylsur2 deficient strain. In addition, GlcCer(d18:2(4E)/16:0(2OH)), which was identified in both WT and Ylsur2 deficient strains, it was found that very small amounts exist compared to GlcCer(d18:2(4E,8E)(9Me)/16:0(2OH)). The bands consistent with the GlcCer(d18:1(4E)/16:0) standard product identified in FIG. 7A were too small to be determined by LC/MS analysis (FIG. 7A and FIG. 7B).

Next, an additional TLC analysis was performed to identify inositol sphingolipid, another final sphingolipid present in Y. lipolytica. Sample preparation and TLC analysis methods were based on S. Tanaka and M. Tani. (2018). The cell pellets of the strains cultured for 8 hours in YPD were recovered and reacted with 350 ul of ethanol/water/diethyl ether/pyridine/15 M ammonia (15:15:5:1:0.018, v/v) at 65° C. for 15 minutes, and then the supernatant was separated, thoroughly dried, and then reacted using 130 ul of monomethylamine (40% in methanol)/water (10:3, v/v) at 53° C. for 1 hour to remove unnecessary phospholipids. The well-dried samples were then prepared by dissolving in 60 ul of chloroform/methanol/water (5:4:1, v/v). Samples extracted from wild-type strains and Ylsur2 deficient strains were dropped into TLC silica gel 60 plates by 15 ul and developed using a solvent consisting of chloroform:methanol:4.2M ammonia (9:7:2, v/v). For comparison, the wild-type S. cerevisiae strain (BY4742) used in the referenced paper was analyzed together. After deployment, copper sulfate (CuSO4) detection method was used for identification, and the differentiation of IPC, MIPC, and M(IP)2C was inferred from the analysis results of the Saccharomyces cerevisiae strain. As a result of TLC analysis, the wild-type Y. lipolytica strain was mainly identified as B type (t18:0/24:0) with phytosphingosine base as the basic backbone or C type (t18:0/24:0(2OH)) with a hydroxyl group attached to the fat chain, while the A type (d18:0/24:0) with the dihydrosphingosine base and the B′ type (d18:0/24:0(2OH)) with the hydroxyl group attached to the fat chain were also slightly detected since phytosphingosine is not produced at all in the case of the Y. lipolytica sur2 deficient strain (FIG. 7C).

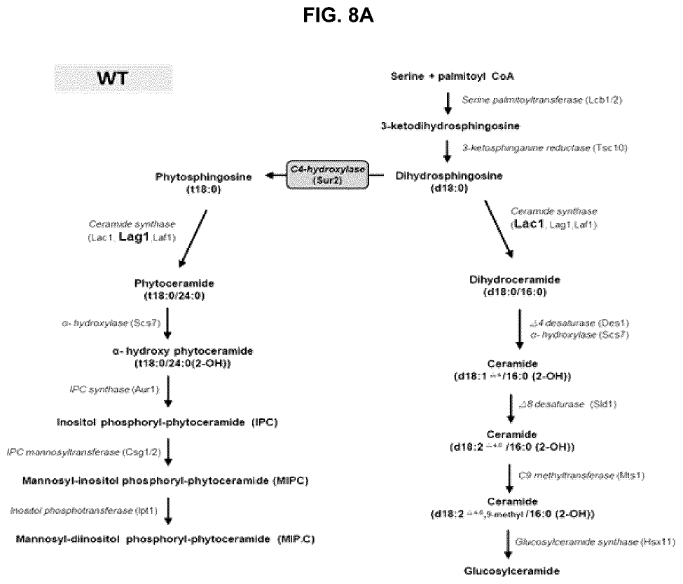

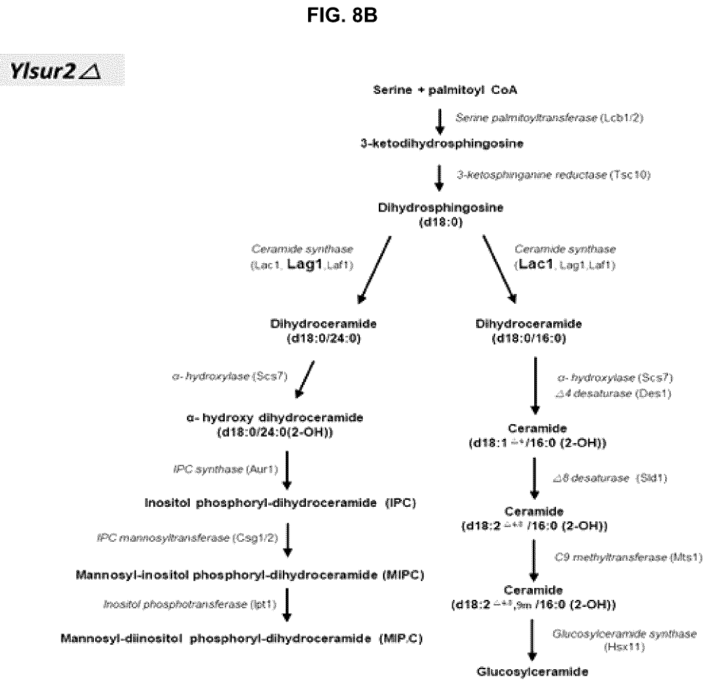

Therefore, based on the TLC and LC/MS results of cell surface samples, the sphingolipid biosynthesis pathway of wild-type Y. lipolytica and sur2 deficient strains is summarized as follows (FIG. 8).

In the case of the wild-type Y. lipolytica strain, most of the inositol sphingolipids are synthesized while having phytoceramides as the backbone, and all types of IPC and MIPC (t18:0/24:0 (20H), C type) have been found in the LC/MS results, suggesting that most of them have a structure with a hydroxyl group (—OH) attached to the fatty acid chain due to Scs7. In addition, it was determined that glucosylceramide is secreted onto a cell surface in a relatively larger amount than inositol sphingolipid, and dihydrosphingosine is synthesized as the basic backbone. Afterwards, A4 desaturase (Des1), α-hydroxylase (Scs7), A8 desaturase (Sld1), and C9 methyltransferase (Mts1) may act to synthesize ceramides of various structures. Finally, glucosylceramide is synthesized by glucosylceramide synthase (Hsx11), and the GlcCer (d18:2(4E,8E)(9Me)/16:0(2OH)) structure produced by the action of all the aforementioned enzymes is present in the highest proportion (FIG. 8A).

On the other hand, in the case of the Y. lipolytica sur2 deficient strain, it may be seen that the deletion of the SUR2 gene, which is responsible for the synthesis of phytosphingosine, would lead to synthesis of MIPC (d18:0/24:0, A type) with dihydrosphingosine as the backbone. On the other hand, considering that a large amount of glucosylceramide is secreted onto the cell surface compared to wild-type strains, it may be inferred that a large amount of dihydrosphingosine is accumulated due to the deletion of the SUR2 gene, and thus the synthesis of glucosylceramide having the same as the basic backbone also increases (FIG. 8B).

### <Example 6> Amplification of Sphingosine Biosynthesis Through Overexpression of Ceramidase Genes in Y. lipolytica Sur2 Deficient Strain

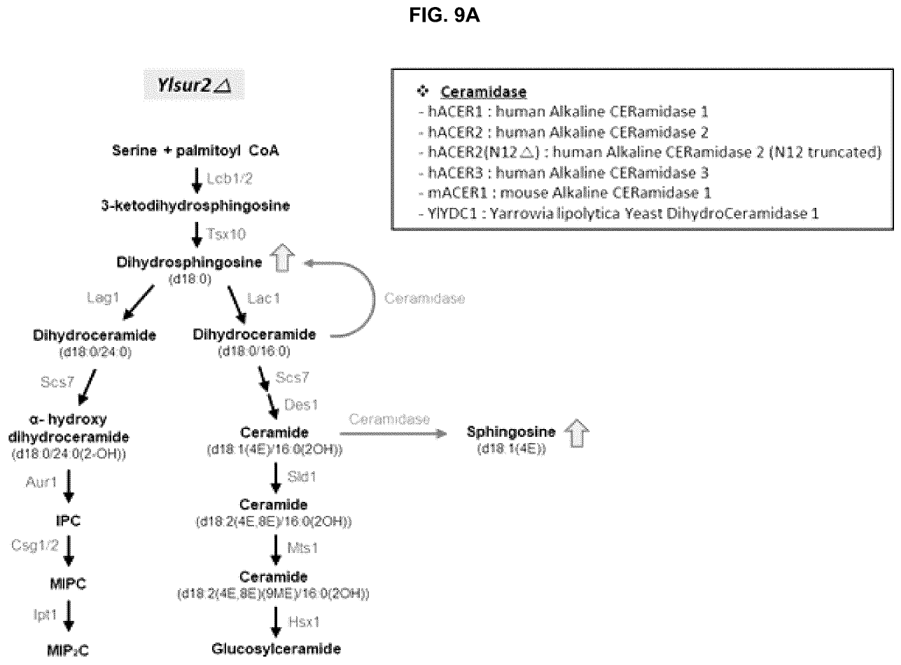

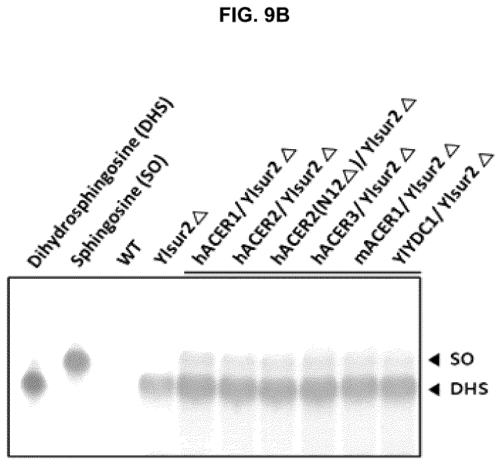

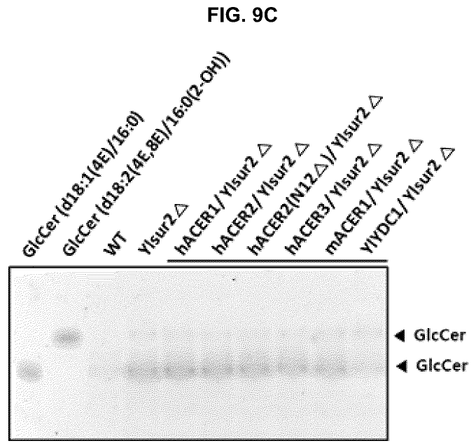

In the Y. lipolytica sur2 deficient strain, the ceramidase gene derived from humans and mice was additionally expressed to amplify sphingosine biosynthesis from ceramide (FIG. 9A). The genes encoding three types of human-derived alkaline ceramides ACER1, ACER2, and ACER3 and the mouse-derived ceramidase ACER1, which are known to produce sphingosine from ceramides, were synthesized through codon optimization in Y. lipolytica. The human-derived alkaline ceramidase genes hACER1 (SEQ ID NO: 5), hACER2 (SEQ ID NO: 6), and hACER3 (SEQ ID NO: 7), the mouse-derived ceramidase gene mACER1 (SEQ ID NO: 8), the human-derived alkaline ceramidase 2 gene hACER2 (N12Δ) (SEQ ID NO: 9) having 12 amino acids removed from the N terminal to induce expression of endoplasmic reticulum, and the DNA fragment having information on the ceramidase gene YIYDC1 (SEQ ID NO: 10) derived from Y. lipolytica were prepared by ligating via PCR and fusion PCR with TEF/promoter and XPR2 terminator using the primers listed in Table 1, and then introduced into the pIMR53 CEN vector to prepare pIMR53-hACER1, pIMR53-hACER2, pIMR53-hACER2(124), pIMR53-hACER3, pIMR53-mACER1, and pIMR53-YIYDC.

Each vector was introduced into the Ylsur2 deficient strain to produce hACER1 Ylsur2 Δ, hACER2 Ylsur2 Δ, hACER2(N12Δ)/Ylsur2 Δ, hACER3/Ylsur2 Δ, mACER2/Ylsur2 Δ, and YIYDC1/Ylsur2 Δ strains (Table 2). Each strain was treated with methanol to be 10 ml/g in pellet samples cultured in SC-U medium for 4 days, and the supernatant obtained by centrifugation was subjected to sonication for 30 minutes to be used for TLC analysis. As a result, the production of dihydrosphingosine as well as sphingosine increased in all strains that were introduced with ceramidase (FIG. 9B). However, TLC for analyzing glucosylceramide showed a decrease in glucosylceramide only in the YIYDC1/Ylsur2 Δ strain (FIG. 9C). Although the accumulation of sphingoid precursors is not more dominant in the Y. lipolytica ceramidase overexpressing strains than other ceramidase overexpressing strains, analysis indicates that the ability to decompose ceramides to produce sphingosine and dihydrosphingosine is more predominant.

### <Example 7> Preparation of Yarrowia lipolytica Sld1 Deficient Strain and Analysis of Growth Features

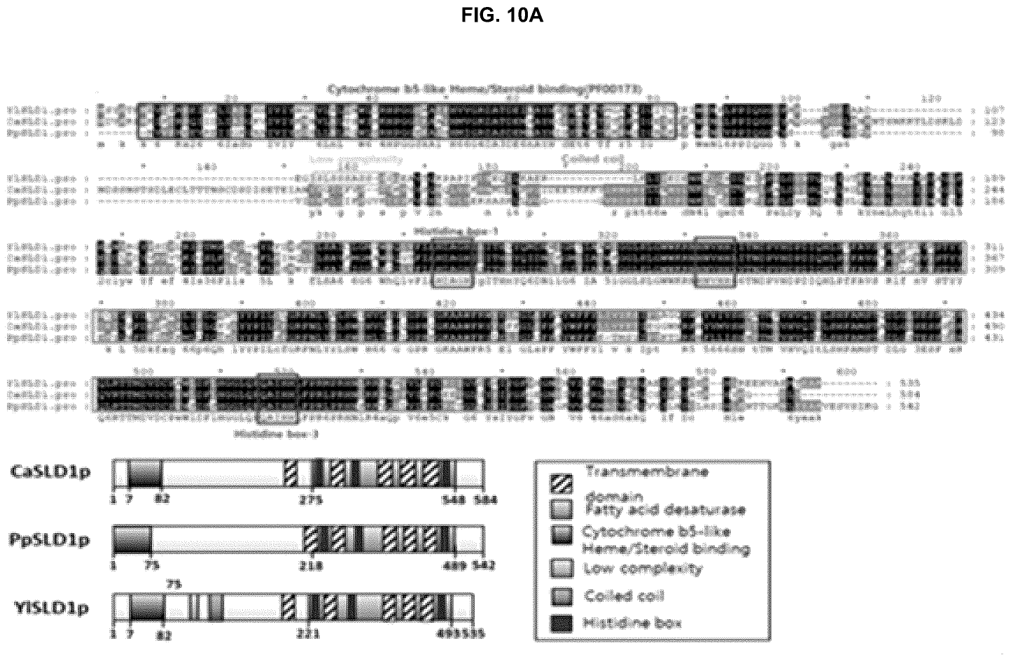

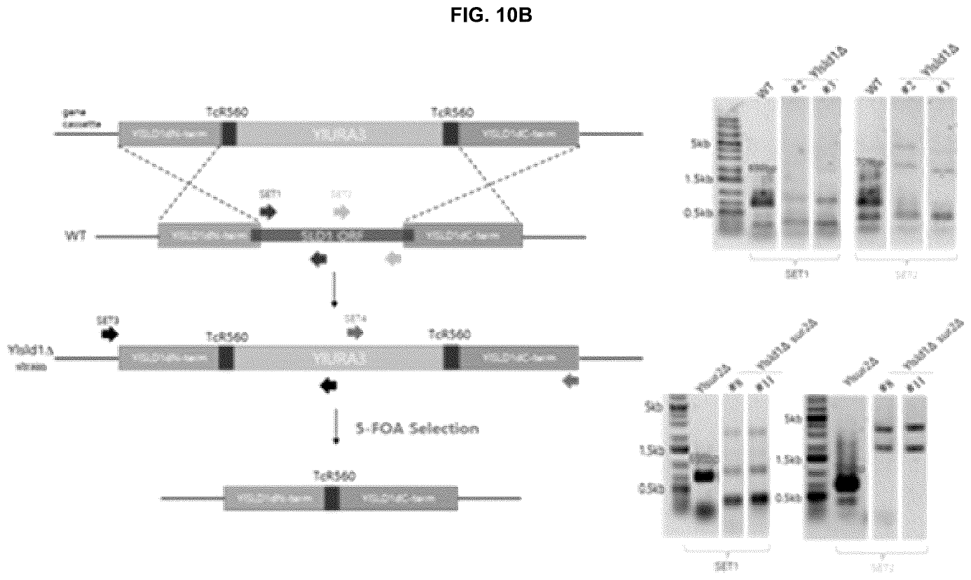

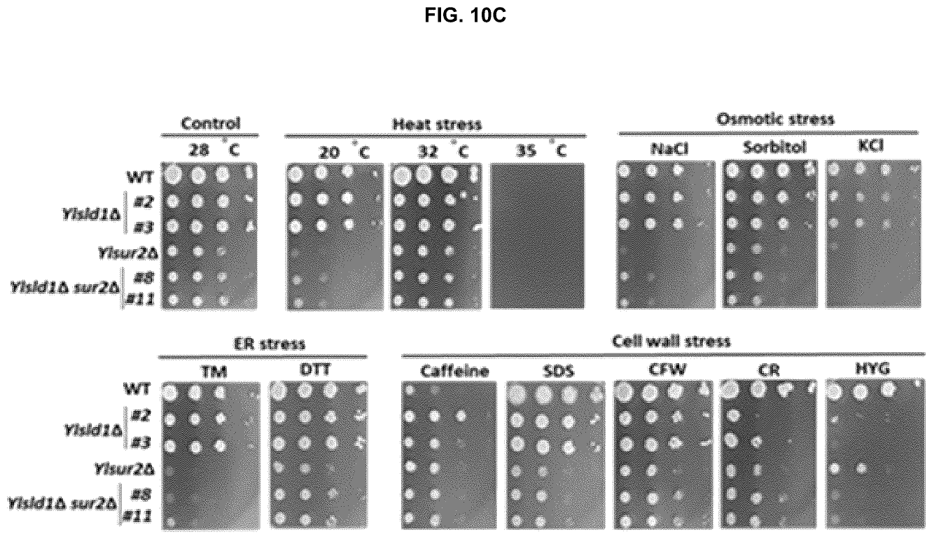

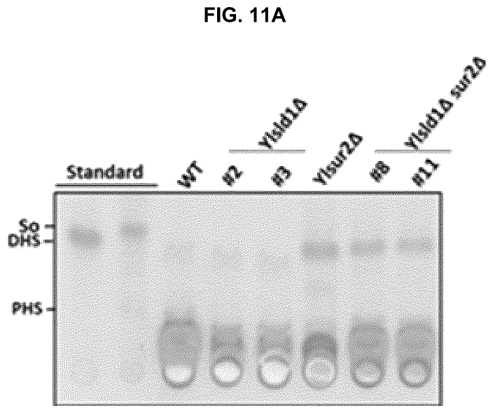

In order to amplify the sphingosine biosynthesis pathway in the Y. lipolytica sur2 deficient strain, the SLD1 gene (SEQ ID NO: 12) encoding the A8 desaturase protein was intended to be additionally disrupted using homologous gene recombinant techniques. The Y. lipolytica Sld1 protein showed slight structural differences, with low complexity and coiled coil structures, unlike Candida albicans and Pichia pastoris. However, cytochrome B5-like heme/steroid binding, fatty acid desaturase, histidine box, and membrane domains were well conserved (FIG. 10A).

To prepare the Y. lipolytica SLD1 gene disruption cassette, the 5′ portion of the SLD1 gene including the SLD1 promoter −869 bp site and the open reading frame (ORF) 152 bp were amplified by PCR, the 3′ portion of the SLD1 gene including the ORF 1,582 bp site and the terminator 955 bp were amplified by PCR, and the 5′ and 3′ portions were ligated by fusion-PCR using the ClaI-NheI-SalI portion which is possessed both by 5′ and 3′ portions, and then introduced into the pT blunt vector to prepare a pTB-SLD1dNC vector. The YIURA3 gene fragment was then amplified by PCR, treated with ClaI/NheI, and ligated to the ClaI/NheI-treated pTB-SLD1dNC vector to prepare a pTB-SLD1dNC-YIURA3(TcR) vector. Afterwards, the cassette was prepared by treating the pTB-SLD1dNC-YIURA3 (TcR) vector with HindIII/NsiI, and the prepared wild-type Y. lipolytica strain and sur2 deficient strain were transformed to produce the Y. lipolytica sld1 deficient strain (Ylsld1Δ) and sld1 sur2 double deficient strain (Ylsld1 Δ sur2Δ) (FIG. 10B).

To investigate the growth features of the Y. lipolytica sld1 deficient strain, it was cultured under YPD medium conditions including inducers of temperature stress (20˜35° C.) and osmotic stress (NaCl, Sorbitol, KCl), inducers of ER stress (Tunicamycin, DTT), and inducers of cell wall stress (Caffeine, SDS, CFW, Congo red, Hygromycin B). As a result of growing for 3 days after dropping onto YPD plates, it was observed that the Ylsld1 deficient strain showed slightly reduced growth even under normal culture conditions and even more stunted growth under temperature stress conditions compared to wild-type strains. In addition, the Ylsld1 deficient strain showed increased susceptibility to Congo red and hygromycin B, which cause cell wall stress, but was also resistant to caffeine-containing media. This feature is considered to be the result of the weakening of cell membranes due to abnormal production of sphingolipids, making the cell wall vulnerable to various stresses to allow antibiotic substances with large molecular weights to easily penetrate into the cells. In contrast, the Ylsld1 sur2 double deficient strain showed a slight recovery in growth compared to the Ylsur2 deficient strain and increased resistance under the stress condition medium. However, further increased sensitivity was shown in some media including KCl and hygromycin B. This is thought to be the result in that the instability caused by the structural changes in the cell membrane due to excessive accumulation of dihydrosphingosine with the deletion of SUR2 was somewhat stabilized by the additional SLD1 deletion to recover the growth (FIG. 10C).

### <Example 8> Identification of Secretion and Production of Sphingosine and Glucosylceramide of Yarrowia lipolytica Sld1 Deficient Strain onto Cell Surface Through TLC and LC/MS Analysis

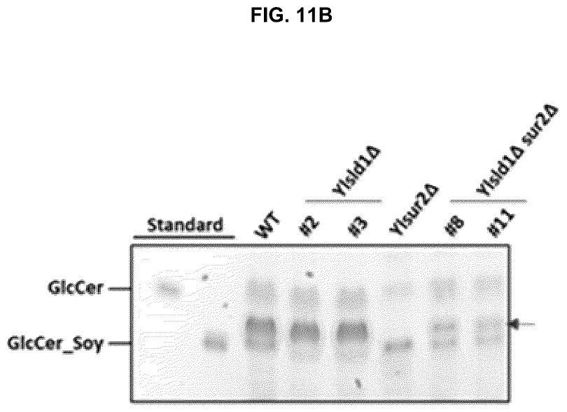

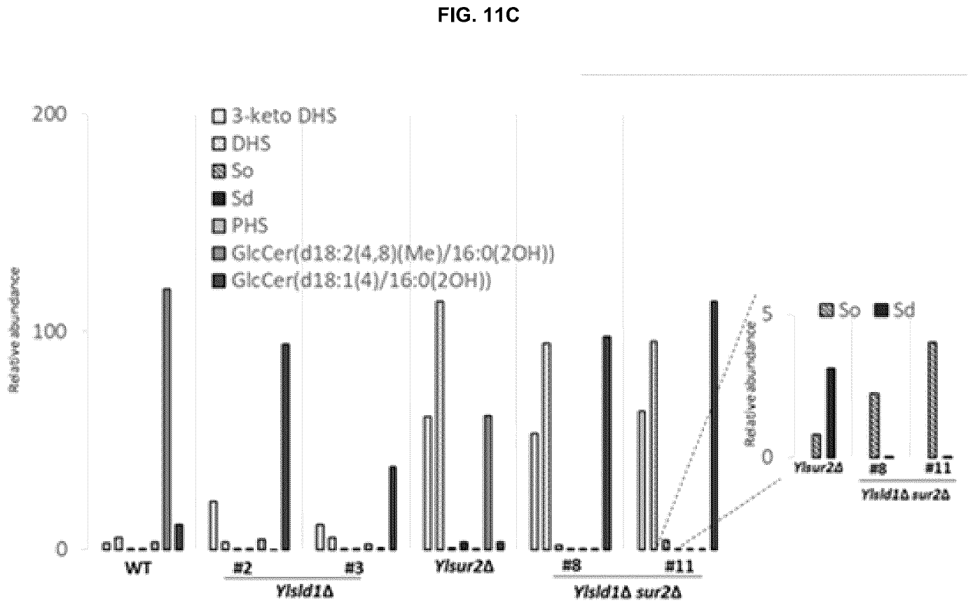

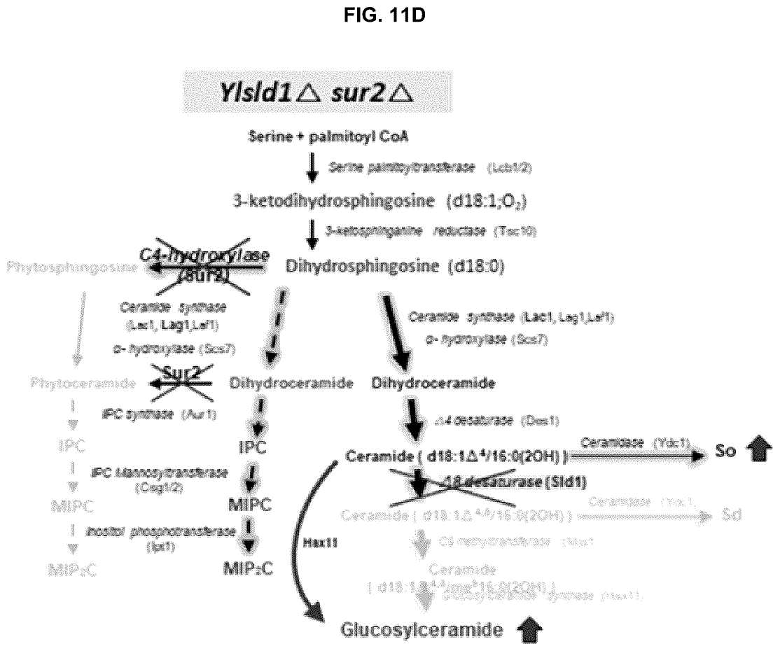

In order to analyze the overall changed lipid components in the Y. lipolytica sld1 deficient strain and the sld1 sur2 double deficient strain, it was intended to identify pure sphingoid bases and glucosylceramides, which are substances at the final stage of the sphingolipid synthesis pathway. Sample preparation was the same as in Example 3, and TLC analysis was performed. As a result of TLC analysis, compared to the Y. lipolytica sur2 deficient strain (Ylsur2 Δ), productivity of dihydrosphingosine was reduced in the Y. lipolytica sld1 sur2 double deficient strain (Ylsld1 Δ sur2Δ) (FIG. 11A). In addition, compared to the Ylsur2 deficient strain, productivity of the final form of glucosylceramide GlcCer(d18:2(4E,8E)9Me/16:0(2OH)) was reduced, and a new type of glucosylceramide bands (red arrows) were generated. The new glucosylceramide has a more similar structure to the human glucosylceramide because it lacks the C8 double band and the C9 methylation formula (FIG. 11B).

For further analysis, as a result of performing LC/MS analysis, it was found that production of sphingadiene (Sd) of the Y. lipolytica sld1 sur2 double deficient strain was blocked, productivity of sphingosine (So) was increased by the decreased amount of sphingadiene, and the amount of dihydrosphingosine was also reduced compared to the sur2 deficient strain. In addition, both the Y. lipolytica sld1 deficient strain and the sld1 sur2 double deficient strain showed a decrease in the amount of glucosylceramide GlcCer (d18:2(4E, 8E)9Me/16:0(2OH)) compared to the control wild-type strain and the Ylsur2 deficient strain, respectively, and an increase in the productivity of glucosylceramide GlcCe (d18:1(4E)/16:0(2OH)) using ceramide from which the C8 double bond generation process by Sld1 and C9 methylation process by Mts1 were omitted, instead (FIG. 11C).

Therefore, based on the TLC and LC/MS results of cell surface samples, the sphinglipid biosynthesis pathway of the Ylsld1 Δ sur2 Δ strain is summarized as follows (FIG. 11D). In the case of dihydrosphingosine that was increased by the deletion of SUR2, it may be seen that an amount of dihydrosphingosine was slightly reduced due to the increase in the ceramide metabolism pathway based on the sphingosine backbone due to the additional deletion of SLD1, and sphingosine was increased as the production pathway for sphingadiene was blocked. In addition, by blocking the formation of a double bond in the eighth sphingosine in the sphingolipid, the amount of the conventional glucosylceramide GlcCer (d18:2(4E, 8E)/16:0(2OH)) was reduced, and a new form of glucosylceramide GlcCer (d18:1(4E)/16:0(2OH)), which has a structure closer to human, was produced as the main glucosylceramide (FIG. 11D). This suggests that the Y. lipolytica SLD1 gene deletion does not completely block the pathway of glucosylceramide production from the ceramide, but rather that glucose may be attached through a different bypass pathway, resulting in the production of finally restructured glucosylceramide.

Having described in detail the specific part of the present disclosure above, it will be apparent to those skilled in the art that the specific description is merely preferred example embodiments and the scope of the present disclosure is not limited thereby. Accordingly, the substantial scope of the present disclosure will be defined by the appended claims and their equivalents.

1. A Yarrowia lipolytica mutant strain in which a SUR2 gene is deleted or expression thereof is suppressed in a Y. lipolytica strain.

2. The Y. lipolytica mutant strain of claim 1, wherein the SUR2 gene is represented by SEQ ID NO: 4.

3. The Y. lipolytica mutant strain of claim 1, wherein the Y. lipolytica mutant strain is a strain deposited under Accession number KCTC 14592BP.

4. The Y. lipolytica mutant strain of claim 1, wherein the Y. lipolytica mutant strain has enhanced secretion and production of dihydrosphingosine or glucosylceramide onto a cell surface.

5. A method of producing dihydrosphingosine or glucosylceramide, comprising culturing the Y. lipolytica mutant strain according to claim 1 in a medium.

6. The method of claim 5, wherein the medium comprises glycerol as a carbon source.

7. The method of claim 5, wherein the method is characterized in that dihydrosphingosine or glucosylceramide are secreted onto a cell surface by means of lipid droplets without additional acetylation process.

8. A Y. lipolytica mutant recombinant strain in which an SLD1 gene encoding Δ 8 desaturase is additionally deleted from the Y. lipolytica mutant strain according to claim 1, with enhanced secretion and production of sphingosine or human glucosylceramide onto a cell surface.

9. The Y. lipolytica mutant recombinant strain of claim 8, wherein the SLD1 gene encoding Δ 8 desaturase is represented by SEQ ID NO: 12.

10. The Y. lipolytica mutant recombinant strain of claim 8, wherein the human-type glucosylceramide is produced as glucosylceramide GlcCer(d18:1(4E)/16:0(20H)).

11. The Y. lipolytica mutant recombinant strain of claim 8, wherein the Y. lipolytica mutant recombinant strain is a strain deposited under Accession number KCTC 14980BP.


SEQUENCE LISTING (56 sequences)

SEQ ID NO: 1 - protein, 458 aa, Yarrowia lipolytica
MDVFFDMNRHFINMAPNFASRRIIYITDDDTPTTNEDDINKTRVRIEDLSHLKVKVEPLLINPSEDKTFDSSKFYALVFNEDTSVEPVEAIDLKQFINKRNVLNRSLFNVKMEIGEGLVVGVRGYLLYAEQKATSTTRKAWVYTGGEKPEIAKLESQAVTIESGRSVDKADLRKTFKFGNDYVPFTEEQLTQIRYFGEPIIRILGFHNSSDFSELFIHSVRSSMFLYPTDEKLVGSIRAFSALYQSLKNKDKMALAWVIVRKGAKPILALLIPSTKEIEGLHMVFLPFTDDIRQEPKTELVSAAPELVDATKNIFTRLRMPGGFESQRYPNPRLQWHYRVVRAMALQEEVPKVPEDKTTPKYRSIDTRVGDAIEEWNKVLQSSSKRPAEDICKAEKKVKSSDAGPPSNEQMQNMVENDIVGKLTVAELRAWGAANNVEPNGSKLKKDWVEVVKKYYGK

SEQ ID NO: 2 - DNA, 1374 bp, Yarrowia lipolytica
  source 1..1374: mol_type = genomic DNA
atggacgtgtttttcgacatgaaccgccattttatcaacatggcacccaacttcgcgtccagacgaatcatctatatcacagacgacgatacccccacgacgaatgaggacgatatcaacaagacacgagttcgaattgaggatctaagccatctcaaggtgaaggtcgagcctcttttgatcaacccttcggaagacaagacgttcgactcctccaaattctacgctcttgtgttcaacgaagacacatctgtggagccggttgaggcgatcgatttgaagcagtttatcaacaaaagaaacgtgctcaatcgatcactgttcaatgtcaaaatggaaatcggagaaggtcttgttgtcggagtaagaggataccttctttatgcggaacaaaaggctacttcaacaacccgaaaggcctgggtttacactggaggtgagaaacccgagattgccaaattagaatcgcaggccgtcactattgaaagtggcagaagcgtggacaaggcagatctgagaaagactttcaagtttggaaatgactatgttcctttcacagaagaacagctgacgcaaatccggtactttggagagccaattattcgaattctcggcttccacaattcctcggacttctccgagctcttcatccacagtgtccgatcgtcaatgttcctatatcccactgatgagaagcttgtgggttcgattcgagccttttcagcactctatcagagtctcaagaacaaggataagatggctctggcctgggttattgtccgcaagggcgccaaacctattctggctcttcttattccttcaactaaggagatcgaaggtcttcatatggtcttcttgccttttacagatgatattcgacaagaaccaaagactgaacttgtgtctgccgcccctgagctcgtggacgcaaccaagaatattttcactcgtctacgcatgcctggcggatttgagtcgcaaagataccccaacccccgtctacagtggcattaccgagttgtacgagccatggcccttcaggaggaggttcccaaggtacccgaagacaagacgacaccaaagtatcggtctattgatactcgagttggtgatgccatcgaggaatggaacaaggtgttgcagagcagctccaagcgacctgcggaggatatctgtaaggctgagaagaaagtcaagagttctgacgcgggccctccgtccaacgagcaaatgcaaaatatggttgagaatgacattgtcggcaagctgaccgtcgcagaactcagggcttggggtgctgctaacaatgttgagcccaatggtagcaagttgaagaaggactgggttgaggtggtcaaaaagtactatgggaag

SEQ ID NO: 3 - protein, 353 aa, Yarrowia lipolytica
MYNGTFNLGPITLTAPNVSSIPPPIYKHSIPPLVSWISDENLALLLPIICYWTSGIMFHIIDQYELLEQYRIHTPEEVTKRNKCTFAEVVRDVIKQHMLQTALGLLLNYFEEPNTTGHEAHDIWTWQVRFGGQTSPAKAWLAAFAYTYLLPFVKISFAFFILDSWQYFLHRAMHQSKWLYKKFHSRHHRLYVPYAIGALYNTALEGVLMDSCGAGLAYMVSGLTTREAIWFFCFSTLKTVDDHCGYCIPWDPFQILFPNNAVYHDIHHQSFGIKTNFSQPFFTFWDRILNTEYHGTRGYVQKQKEITAQKYKEWVESRSGSSSEDENKAKATEVAGKSTGASTSEQVVSSRLE

SEQ ID NO: 4 - DNA, 1059 bp, Yarrowia lipolytica
  source 1..1059: mol_type = genomic DNA
atgtacaacggcaccttcaatctcggcccaataacgctgacggcccccaacgtgtcgtcgataccgcctcctatctacaaacactccattccaccactcgtgtcatggatctcggacgagaatctcgcgctgctgctgcccatcatctgctactggacgtcgggcatcatgttccacatcatcgaccagtacgagctgctcgaacagtaccggatccacactcctgaggaggtgaccaagcgcaacaagtgcacgtttgcggaggttgttcgagacgttatcaagcaacacatgctccagacggctctgggcctgctactcaactactttgaggagcccaacacgactggccatgaagctcacgacatttggacgtggcaggttcggtttggaggacagacgagccccgcaaaggcgtggctcgcggcgttcgcctacacctatctgctgccctttgtcaaaatctcctttgccttcttcattctcgactcgtggcagtactttctgcatcgggccatgcaccagagcaaatggttatacaaaaagttccactccagacaccacagactctatgtgccctacgccattggagcgctgtacaacactgctctggagggcgtgctgatggactcgtgtggagccggcctggcatacatggtttcgggcctgactacccgagaagccatctggttcttctgcttctccacgctcaagactgtcgatgaccactgcggctactgcattccctgggacccgttccagatcttgttccccaacaatgccgtgtaccacgacattcaccatcagagctttggcatcaagaccaacttttcgcagcccttcttcaccttctgggaccgcattctcaacacggagtaccacggcacccggggctacgtgcagaagcagaaggagatcacggcccaaaagtacaaggagtgggttgagtctcgaagcggctcttcgtccgaggatgagaacaaggccaaggccaccgaggtggctggcaagtcgactggagcttctacttccgagcaggtggtttcttctcgacttgag

SEQ ID NO: 5 - DNA, 792 bp, unidentified
  source 1..792: mol_type = genomic DNA; note = Human
atgccttcgatcttcgcataccaatcctctgaggtggattggtgtgaatccaacttccagtattccgaactggtggccgagttttacaacacgttctccaacatccctttcttcatctttggaccccttatgatgctcctgatgcatccttacgctcagaagcgttctcggtacatttatgttgtctgggtcctctttatgattatcggccttttctctatgtactttcacatgacactgtccttccttggacaactcctggacgagattgccattctctggctgctcggaagcggctattccatctggatgcctagatgctactttccttccttccttggtggtaacagatcgcagttcattagactggttttcattactaccgttgtttccactctgctctcctttctgcgacccactgtgaacgcttatgctcttaattccattgcactgcacattctttacattgtctgtcaagaatatcgtaagacaagcaataaggagctccggcacctgattgaagtttctgttgttctgtgggccgtggctctgacatcctggatctccgaccggctcctctgctcgttctggcaacggattcattttttttacctccactcgatttggcatgtcctcatctctattacttttccttacggaatggttaccatggcccttgttgacgccaactacgagatgcctggtgagacactgaaggtgagatattggcccagagacagctggcccgtcggactgccttatgtggaaattcggggagatgacaaggattgt

SEQ ID NO: 6 - DNA, 825 bp, unidentified
  source 1..825: mol_type = genomic DNA; note = Human
atgggagcacctcattggtgggatcagcttcaagctggctcgtctgaggtggactggtgcgaagataactataccatcgttcccgcaattgcagagttctacaacactatcagcaacgtgctctttttcatccttccccctatttgtatgtgtctctttagacaatatgcaacctgttttaattccggaatttatctcatctggaccctgctggttgtggttggaattggtagcgtctacttccacgcaactctctcttttcttggccaaatgctggatgaactcgctgttctctgggtgcttatgtgcgctctggccatgtggttcccccgacgttatcttcccaaaatctttcgtaatgacagaggtagattcaaagttgttgtctctgtcctgtcggcagtcactacatgcctggctttcgtcaagcccgcaattaacaacatcagcctgatgacacttggagtgccctgcactgctctgcttatcgcagaactgaagcgttgcgataacatgcgggtgttcaagcttggcctcttctctggtctttggtggaccctcgcactgttttgttggatttcggacagagcattctgcgaacttctcagctcttttaacttcccctatctccattgcatgtggcatattctgatttgtcttgctgcctatcttggctgtgtctgcttcgcatatttcgatgcagctagcgaaatccctgaacaaggtcccgtgattaaattttggcctaatgagaaatgggcattcattggagttccctacgtctctcttctttgcgctaacaaaaagagcagcgttaagattacc

SEQ ID NO: 7 - DNA, 801 bp, unidentified
  source 1..801: mol_type = genomic DNA; note = Human
atggcccccgccgcagatcgagagggctattggggacctacgacttcgacccttgattggtgcgaagaaaattactccgtcacctggtatattgctgagttctggaataccgttagcaacctcattatgattattccccccatgttcggcgccgttcagagcgtgcgggacggcctcgaaaagcgatatatcgcctcttatctcgctcttacagtcgttggtatgggttcttggtgctttcacatgacgctcaaatacgagatgcaactgctggacgagctgcccatgatttactcgtgctgtattttcgtgtattgcatgttcgaatgtttcaagattaaaaattcggtcaactatcacctgctgtttactctcgtgctgttttctctcattgtgacaacggtctaccttaaggttaaagagcccatttttcaccaagtgatgtacggtatgcttgtgtttacgctcgtgcttcgttccatttacattgtgacttgggtgtatccctggcttcgtggcctgggatacacgtctctgggtatctttctgcttggcttcctgttctggaatattgataacattttttgtgagtccctcagaaactttcgaaagaaggtcccccccattattggtatcacaactcagttccacgcatggtggcatatccttactggtctgggttcgtaccttcatattcttttttccctctatacgagaacgctctatctccgataccggcccaaggtgaagtttctctttggcatttggcctgtgattctctttgaacctcttcgaaaacac

SEQ ID NO: 8 - DNA, 819 bp, unidentified
  source 1..819: mol_type = genomic DNA; note = Mouse
atgcacgtccctggaacgagagccaaaatgagcagcatctttgcctatcagtccagcgaagtcgattggtgcgagtcgaattttcaacactctgagctggttgccgagttttacaatacgttctcgaatgtcttttttctcattttcggacctctgatgatgtttcttatgcatccttacgcacaaaaacggactagatgtttctacggcgttagcgtgctttttatgctgattggtctcttctccatgtatttccacatgactctgtcttttcttggtcagcttctggatgagatttctatcctgtggctccttgcatcgggatactctgtttggctgccccgatgttatttccccaagtttgtcaagggaaaccgtttctacttttcgtgtctggtcaccatcacgacgattatttctacatttctgacatttgtcaaacccactgtcaatgcatacgcacttaattccattgcaattcatatcctgtacattgtgagaacggaatacaaaaaaatccgggacgacgacctccgacacctgattgcagttagcgttgtcctctgggcagcagcccttacaagctggattagcgaccgagttctgtgctctttctggcagagaatccatttttattatcttcattcgatttggcatgttctcatttcgatcacttttccctatggaatcgtgaccatggcactggttgatgctaagtacgagatgcccgataagacgctcaaggttcactattggcccagagactcctgggtcattggtcttccttatgtcgagatccaggaaaatgataagaactgt

SEQ ID NO: 9 - DNA, 789 bp, unidentified
  source 1..789: mol_type = genomic DNA; note = Human
atgtcgtctgaggtggactggtgcgaagataactataccatcgttcccgcaattgcagagttctacaacactatcagcaacgtgctctttttcatccttccccctatttgtatgtgtctctttagacaatatgcaacctgttttaattccggaatttatctcatctggaccctgctggttgtggttggaattggtagcgtctacttccacgcaactctctcttttcttggccaaatgctggatgaactcgctgttctctgggtgcttatgtgcgctctggccatgtggttcccccgacgttatcttcccaaaatctttcgtaatgacagaggtagattcaaagttgttgtctctgtcctgtcggcagtcactacatgcctggctttcgtcaagcccgcaattaacaacatcagcctgatgacacttggagtgccctgcactgctctgcttatcgcagaactgaagcgttgcgataacatgcgggtgttcaagcttggcctcttctctggtctttggtggaccctcgcactgttttgttggatttcggacagagcattctgcgaacttctcagctcttttaacttcccctatctccattgcatgtggcatattctgatttgtcttgctgcctatcttggctgtgtctgcttcgcatatttcgatgcagctagcgaaatccctgaacaaggtcccgtgattaaattttggcctaatgagaaatgggcattcattggagttccctacgtctctcttctttgcgctaacaaaaagagcagcgttaagattacc

SEQ ID NO: 10 - DNA, 960 bp, Yarrowia lipolytica
  source 1..960: mol_type = genomic DNA
atgctacctggaggaatcccatacccaccacccgtggacaacggctactggggccccacgacatcgaccattgactggtgcgaggaaaactacgtggtcagcaaatacgtggcggaaatcatgaacaccaccacaaacgccgtgttcatgatcatggcgctgtacaccatcattaacgtgtaccgggagaaacaccaccccaccatcattttcgccgccattggctttttcattgtgggatttggttcctggatgttccacatgacgctgtggtacgagttccagctgctggatgagctacccatgatctacgccacctgcgtgccgttgtacattgtcttctccaacaaaaagagcaaccacttcaagacgctgctgggcgtcggaatcgccgccggtgccctgttgctcacagccatctacctgcacaacaagaaccccaccttccaccaggccgcctacggtattctcaacttcattgtcattggaaagtctgtggctctgactaaggcctacatttccgaccagaagaccaagaacctcttctggcgactgctcgcccttggcctcttctccttcctctttggctactttctgtggaacctggacatccacctgtgcaaccagtggatcaagatccgacgagaagtcggcctgccctacggtctggttatcgagggccatgcctggtggcacattttcaccggactcggagtctacatctacattgtctacctgtgctacctgcaggtcttcctggccaagcgacagcacctctaccactttctctggtggtgcggcttcttcccccacgtggatctgctgcctgaagccaagtctctgtggaacaagctgggccgagctcccaccgaaactgagatggccgaggtcaattcgctcagtggagtcaagcgagtctcctccatccatgccaagagctcttctgttaaccatcacaac

SEQ ID NO: 11 - protein, 535 aa, Yarrowia lipolytica
MSYKTRKEVLPRSEIEALIADGHHIVIYNGKVLKLDNWLDRHPGGDMAIFHVVGRDATDEINAYHSDEALATMGAYQIGITGEKIWENFLPPIQGGVFRKRGSIAADEGISLSSSASSSSQEAVTQKPAPIFTNEQILKAERLKLIEEDENRQLQHDLEIFPSLDYNTQTEIGTKYKALYKRVEDAGLFEPNYRGYFSDFLRISTMFSLSWYLFDKSMFLSAICLGMGWHQLVFIAHDAGHIGITHGYQMDNWIGMTIAAWIGGLSLGWWKRNHNVHHIITNDPVHDPDIQHLPVFAVSNRFFSSVKSTYYNCVLKYDIFARYLIPLQHMTYYPILCLGRFNLYALSWGHVLLGRGPRHGKAAWFRWYELMGLSFFAYWFFYLIVYKSIPTAGQRWMYVIVSHVATMPVHVQITLSHFAMSTQDLGLSESFAQRQLRTTMDVDCPAWLDFLHGGLQFQAIHHLFPRMPRHNLRRAQRYVLEYCKDIGVEYHIYGFVHGNQHVIGKLGEIAKQAEIFRDCERHLREEHVAEMTEAK

SEQ ID NO: 12 - DNA, 1608 bp, Yarrowia lipolytica
  source 1..1608: mol_type = genomic DNA
atgtcctacaagacccgcaaagaggtgcttccgcggtcggaaattgaggcgctcatcgccgacggccaccacattgtcatctacaacggcaaggtgctcaagctggacaactggctcgatcgtcaccctggaggagacatggccattttccacgtggttggacgggacgccaccgacgaaatcaacgcataccacagcgacgaggccctggccaccatgggcgcctaccagattggaatcactggcgaaaaaatctgggaaaactttctgccacctatccagggaggagtgtttcgaaaacggggcagtattgctgcagatgagggcatttcgctgtccagcagcgcatccagctcatcacaggaggcagtgactcagaaacccgcgcccatttttacgaacgaacaaattctcaaggcggaaagacttaagctcatcgaggaggacgagaacagacagctccagcatgaccttgaaatctttccctctctcgactacaacacccagacggagattggaacaaagtacaaggccctgtacaagcgtgtcgaggacgctggcttgtttgaacccaattacagaggctacttcagtgactttcttcgaatctctaccatgttcagtctgtcatggtacctgtttgacaagtccatgttcctctccgccatttgtctgggcatgggctggcaccagctagtcttcattgctcatgatgccggccacattggtatcacccatggctaccagatggacaactggatcggaatgaccattgccgcttggattggaggtctgtctttgggctggtggaagcgaaaccacaacgtgcatcacattattaccaacgaccctgtgcacgaccccgatatccagcatcttcccgtatttgctgtatccaaccgattcttctcgtcagtcaagtccacctactataactgtgtgctcaagtacgacatcttcgctcgatacctcattcccttgcagcacatgacctactaccccattttgtgcctgggtcgattcaacctttacgctctgtcatggggccatgtgcttcttggaagaggccctcgacacggtaaggctgcgtggttccgatggtacgagctcatgggactctccttcttcgcgtactggttcttctacctcattgtgtacaagagcattcccactgccggccagcgatggatgtatgtaattgtgtcgcacgttgctactatgcctgttcatgtgcaaatcactctgtcgcactttgctatgtccacccaggacctgggtctgtcggagtcctttgcccagcgacaactgcgaaccactatggacgtggattgtcctgcctggctcgacttcttgcatggaggtctgcagttccaggccatccaccacctgttccctcgaatgccccgacacaacttgcgaagggcacagcggtacgtgcttgagtactgcaaagatattggcgtggagtaccatatctacggctttgtgcacggcaaccagcacgtgattggtaagctgggcgagattgccaagcaagccgaaatcttccgtgactgtgagcgacatcttcgagaggaacatgttgctgagatgactgaggccaagtag

SEQ ID NO: 13 - DNA, 22 bp, synthetic construct
  source 1..22: mol_type = other DNA
gagcggtttgctcgaaaccagt

SEQ ID NO: 14 - DNA, 54 bp, synthetic construct
  source 1..54: mol_type = other DNA
tacagtcgacgcgtgggcgtatcgattatcgtcgtctgtgatatagatgattcg

SEQ ID NO: 15 - DNA, 46 bp, synthetic construct
  source 1..46: mol_type = other DNA
cgatacgcccacgcgtcgactgtaaggctgagaagaaagtcaagag

SEQ ID NO: 16 - DNA, 21 bp, synthetic construct
  source 1..21: mol_type = other DNA
gagcggtttgctcgaaaccag

SEQ ID NO: 17 - DNA, 18 bp, synthetic construct
  source 1..18: mol_type = other DNA
cagctcggccagcatgag

SEQ ID NO: 18 - DNA, 21 bp, synthetic construct
  source 1..21: mol_type = other DNA
ccaacctgtgtgcttctctgg

SEQ ID NO: 19 - DNA, 20 bp, synthetic construct
  source 1..20: mol_type = other DNA
cgaccgaaaatgtgcaagcg

SEQ ID NO: 20 - DNA, 45 bp, synthetic construct
  source 1..45: mol_type = other DNA
acgagctagccgcgcgacgcgtcgagtggtggaatggagtgtttg

SEQ ID NO: 21 - DNA, 31 bp, synthetic construct
  source 1..31: mol_type = other DNA
gaacagtcgactggagcttctacttccgagc

SEQ ID NO: 22 - DNA, 24 bp, synthetic construct
  source 1..24: mol_type = other DNA
catgcgtcctctcctccacgattc

SEQ ID NO: 23 - DNA, 35 bp, synthetic construct
  source 1..35: mol_type = other DNA
cgcgcggctagctcgtcgcctgagtcatcatttat

SEQ ID NO: 24 - DNA, 35 bp, synthetic construct
  source 1..35: mol_type = other DNA
gctccagtcgactgttcggaaatcaacggatgctc

SEQ ID NO: 25 - DNA, 78 bp, synthetic construct
  source 1..78: mol_type = other DNA
tcaccccgcgtccttcttcgggcagtcgcgcttttctccggcttctgcggaaattgaagctctaatttgtgagtttag

SEQ ID NO: 26 - DNA, 84 bp, synthetic construct
  source 1..84: mol_type = other DNA
ctcatcagttttcgaagatttcaagaaagtccgaagaacatttaagaccttttcaattcaattcatcattttttttttattctt

SEQ ID NO: 27 - DNA, 29 bp, synthetic construct
  source 1..29: mol_type = other DNA
ccggaattcagagaccgggttggcggcgt

SEQ ID NO: 28 - DNA, 50 bp, synthetic construct
  source 1..50: mol_type = other DNA
tgcgaagatcgaaggcattttgaatgattcttatactcagaaggaaatgc

SEQ ID NO: 29 - DNA, 46 bp, synthetic construct
  source 1..46: mol_type = other DNA
gagtataagaatcattcaaaatgccttcgatcttcgcataccaatc

SEQ ID NO: 30 - DNA, 46 bp, synthetic construct
  source 1..46: mol_type = other DNA
atcaccggcaaactatctgtctaacaatccttgtcatctccccgaa

SEQ ID NO: 31 - DNA, 53 bp, synthetic construct
  source 1..53: mol_type = other DNA
ggggagatgacaaggattgttagacagatagtttgccggtgataattctctta

SEQ ID NO: 32 - DNA, 41 bp, synthetic construct
  source 1..41: mol_type = other DNA
ctagtctagaggggggctagccacgggcatctcacttgcgc

SEQ ID NO: 33 - DNA, 52 bp, synthetic construct
  source 1..52: mol_type = other DNA
caccaatgaggtgctcccattttgaatgattcttatactcagaaggaaatgc

SEQ ID NO: 34 - DNA, 41 bp, synthetic construct
  source 1..41: mol_type = other DNA
gagtataagaatcattcaaaatgggagcacctcattggtgg

SEQ ID NO: 35 - DNA, 52 bp, synthetic construct
  source 1..52: mol_type = other DNA
atcaccggcaaactatctgtctaggtaatcttaacgctgctctttttgttag

SEQ ID NO: 36 - DNA, 53 bp, synthetic construct
  source 1..53: mol_type = other DNA
agagcagcgttaagattacctagacagatagtttgccggtgataattctctta

SEQ ID NO: 37 - DNA, 52 bp, synthetic construct
  source 1..52: mol_type = other DNA
cagtccacctcagacgacattttgaatgattcttatactcagaaggaaatgc

SEQ ID NO: 38 - DNA, 41 bp, synthetic construct
  source 1..41: mol_type = other DNA
gagtataagaatcattcaaaatgtcgtctgaggtggactgg

SEQ ID NO: 39 - DNA, 46 bp, synthetic construct
  source 1..46: mol_type = other DNA
caccggcaaactatctgtctaggtaatcttaacgctgctctttttg

SEQ ID NO: 40 - DNA, 52 bp, synthetic construct
  source 1..52: mol_type = other DNA
gagcagcgttaagattacctagacagatagtttgccggtgataattctctta

SEQ ID NO: 41 - DNA, 52 bp, synthetic construct
  source 1..52: mol_type = other DNA
cgatctgcggcgggggccattttgaatgattcttatactcagaaggaaatgc

SEQ ID NO: 42 - DNA, 40 bp, synthetic construct
  source 1..40: mol_type = other DNA
gagtataagaatcattcaaaatggcccccgccgcagatcg

SEQ ID NO: 43 - DNA, 51 bp, synthetic construct
  source 1..51: mol_type = other DNA
atcaccggcaaactatctgtctagtgttttcgaagaggttcaaagagaatc

SEQ ID NO: 44 - DNA, 55 bp, synthetic construct
  source 1..55: mol_type = other DNA
ctttgaacctcttcgaaaacactagacagatagtttgccggtgataattctctta

SEQ ID NO: 45 - DNA, 47 bp, synthetic construct
  source 1..47: mol_type = other DNA
tccagggacgtgcattttgaatgattcttatactcagaaggaaatgc

SEQ ID NO: 46 - DNA, 40 bp, synthetic construct
  source 1..40: mol_type = other DNA
gagtataagaatcattcaaaatgcacgtccctggaacgag

SEQ ID NO: 47 - DNA, 50 bp, synthetic construct
  source 1..50: mol_type = other DNA
atcaccggcaaactatctgtctaacagttcttatcattttcctggatctc

SEQ ID NO: 48 - DNA, 52 bp, synthetic construct
  source 1..52: mol_type = other DNA
ggaaaatgataagaactgttagacagatagtttgccggtgataattctctta

SEQ ID NO: 49 - DNA, 58 bp, synthetic construct
  source 1..58: mol_type = other DNA
gggattcctccaggtagcattttgaatgattcttatactcagaaggaaatgcttaacg

SEQ ID NO: 50 - DNA, 45 bp, synthetic construct
  source 1..45: mol_type = other DNA
gagtataagaatcattcaaaatgctacctggaggaatcccatacc

SEQ ID NO: 51 - DNA, 41 bp, synthetic construct
  source 1..41: mol_type = other DNA
atcaccggcaaactatctgtctagttgtgatggttaacaga

SEQ ID NO: 52 - DNA, 52 bp, synthetic construct
  source 1..52: mol_type = other DNA
ctgttaaccatcacaactagacagatagtttgccggtgataattctcttaac

SEQ ID NO: 53 - DNA, 22 bp, synthetic construct
  source 1..22: mol_type = other DNA
atgcagacggataggtggtggg

SEQ ID NO: 54 - DNA, 56 bp, synthetic construct
  source 1..56: mol_type = other DNA
ctcaagctggacaactggctcgatggtagctacttacgcgatcgtagggccagctg

SEQ ID NO: 55 - DNA, 54 bp, synthetic construct
  source 1..54: mol_type = other DNA
ccatcgatgaatgcgctagcatcccggtcgacgtgactgtgagcgacatcttcg

SEQ ID NO: 56 - DNA, 24 bp, synthetic construct
  source 1..24: mol_type = other DNA
cggctaaccagaaacggtcctatc